(19) United States
(12) Patent Application Publication
(10) Pub. No.: US 2004/0224330 A1
(43) Pub. Date: Nov. 11, 2004

(54) Nucleic acid indexing

(76) Inventor: Liyan He, Chandler, AZ (US)

Correspondence Address: MORRISON & FOERSTER LLP, 755 PAGE MILL RD, PALO ALTO, CA, 94304-1018, US

(21) Appl. No.: 10/759,372

(22) Filed: Jan. 15, 2004

Related U.S. Application Data
(60) Provisional application No. 60/440,044, filed on Jan. 15, 2003.
(60) Provisional application No. 60/444,632, filed on Feb. 4, 2003.

(51) Int. Cl. C12Q 1/68; C07H 21/04

(52) U.S. Cl. 435/6; 536/25.4

(57) ABSTRACT

The present invention describes methods to construct circularization indexers and use them to selectively circularize, purify, and manipulate nucleic acids from a complex nucleic acid mixture. An assay is provided to detect nucleic acid without any amplification. Kits for preparing indexing nucleic acid targets and for high-level multiplexing DNA amplification are provided. The present invention also describes a method to construct a nucleic acids amplification assembly, in which the nucleic acid amplification is self-primed without the need for external primers. In this method, double stranded oligonucleotides with two cohesive ends (C-indexers) are used to circularize double-stranded or single-stranded nucleic acid targets in such a way that one of the strands is completely circularized while the other strand either contains a nick (if the targets are double-stranded) or is only hybridized to part of the targets (if the targets are single-stranded). At the presence of a polymerase, the un-circularized-strand can serve as primer to amplify the circularized strand without any external primer.

### CROSS-REFERENCE TO RELATED APPLICATIONS

This application claims the priority benefit of provisional patent applications U.S. Ser. No. 60/440,044, filed Jan. 15, 2003; and U.S. Ser. No. 60/444,632, filed Feb. 4, 2003; all of which are incorporated herein by reference in their entirety.

### STATEMENT REGARDING FEDERALLY SPONSORED RESEARCH OR DEVELOPMENT

Not applicable.

### FIELD OF THE INVENTION

This invention relates to methods for indexing nucleic acids useful for identifying, isolating, mapping, amplifying target nucleic acids.

### BACKGROUND OF THE INVENTION

It is known in the art of molecular biology that molecular cloning and polymerase chain reaction (PCR) or modifications of the PCR are the most commonly used method for amplifying nucleic acid. The cloning step involves the creation of a gene library by fragmenting or restricting, using suitable restriction enzymes, the genome of an organism, which comprises the starting material for the sought-after gene. These DNA fragments are then inserted into a suitable cloning vehicle, e.g. plasmid DNA (Cohen et al., 1973; Tanaka and Weisblum, 1975) or lambda phage DNA (Thomas et al., 1974; Murray, N. E. and Murray, K., 1974). The hybrid recombinant DNA-cloning vehicles so formed are then introduced into host cells, e.g. E. coli cells, by transformation and cloned by a suitable technique such as single colony isolation or plaque formation.

In most of the cloning methods, two different DNA molecules are cut by the same restriction endonuclease to produce identical cohesive ends. The DNA molecules are annealed to one another and then covalently joined by DNA ligase. This method limits the size and kind of DNA fragments that can be cloned since it requires intrinsic advantageous restriction endonuclease cleavage sites on both ends of the DNA fragment that one is interested in. For example, if one wants to clone a small DNA fragment such as a promoter (e.g., an RNA polymerase protected fragment), the nearest restriction endonuclease sites may be relatively distant, and thus extraneous DNA sequences must be included in the cloned DNA. Many DNA fragments cannot be cloned by this method because of the lack of a suitable restriction enzyme for producing molecules with appropriate cohesive ends.

The requirement for advantageous restriction endonuclease cleavage sites in nucleic acid of interest is circumvented by a DNA Adaptor technique (Wu et al, U.S. Pat. No. 4,321,365). These adaptor molecules are either single-stranded or double-stranded oligonucleotides. If double-stranded, adaptor molecules may have either one protruding nucleotide sequence which is a recognition site for a restriction endonuclease at one end of the duplex or two protruding nucleotide sequences which are recognition sites for the same or different restriction endonucleases at opposite ends of the duplex. Attaching the adaptors to both ends of DNAs to be amplified or to the ends of the cloning vehicles creates a flexible cloning system. This provides a tool for molecular cloning as the same adaptor molecule may be used to introduce any double stranded DNA into cloning vehicles at specific sites.

PCR can be used to isolate and amplify DNA sequences of interest lying between two identified and unique primer sequences (Saiki et al. 1985; Mullis & Faloona 1987; Mullis, U.S. Pat. No. 4,683,202). This technique avoids conventional molecular cloning techniques and can isolate and amplify the selected sequence in a much easier fashion. One identified shortcoming of PCR is that it requires prior knowledge of the nucleic acid sequences flanking the region of interest. This limits it to targets that have already been the subject of investigation at least to the extent of obtaining the necessary flanking sequence information. A second disadvantage is that the requirement of two primers per target also limits the number of targets that can be amplified simultaneously. For non-repetitive analyses of large numbers of different targets, the cost of two unique primers required per target may become prohibitive.

Rolling circle amplification (RCA) was developed to amplify DNA at isothermal condition (Fire, A. & Xu, S-Q, 1995; Liu, D. et al, 1996). In this reaction, DNA polymerase extends a primer hybridized to the circular template, generating a strand that contains multiple head-to-tail copies of the template sequence. Zhang et al. (1998) and Lizardi et al. (1998) have extended this method further by using a primer to initiate copying of the circular template and another primer to copy the RCA product. These procedures provide exponential amplification and generate dsDNA products.

RCA has been used to generate signal amplification for detecting nucleic acids (Lizardi et al., 1998; Zhong et al, 2001; Nallur et al., 2001), single nucleotide polymorphisms (Nilsson et al., 1997), and proteins (Schweitzer et al., 2000). In these studies, either a padlock probe or a circularized oligonucleotide is used as the template for RCA reactions. Amplification can be carried out both in solution and on various surfaces (Nallur et al., 2001). When used with surface-anchored probes, RCA generates enough signals to detect small oligonucleotide hybridized to genomic DNA targets and to visualize point mutations in cultured cell lines and stretched DNA fibers (Zhong et al., 2001). The current RCA applications are limited by the need to synthesize and circularize long DNA probes.

Sabanayagam, et al. (U.S. Pat. No. 6,284,497) described a procedure to amplify circularized DNA targets on DNA array containing immobilized primers. This invention enables high-level multiplexing detection of DNA targets on high-density array without the need for expensive long sequence anchor probes and circular probes. It extended the potential applications of RCA on high-density array. For example, when the circularized DNA targets hybridized to a high-density array containing immobilized allele-specific primers, RCA reaction can be used to determine the genotype of the target DNA without any pre-amplification. Only those gene-specific primers whose 3′ end is complementary to the allele presented in the target will be amplified and detected. RCA circularized DNA targets has great potential because it will drastically reduce the cost of a wide range of nucleic acid analyses. However, Sabanayagam et al. used a bridge primer to circularize DNA target. The bridge primer has to be gene specific for each target.

Clearly, there is a great need for a high-throughput method for preparing circularized DNA targets at low cost.

Type IIS restriction endonucleases cleave DNA asymmetrically at defined distances from their recognition sites, and Interrupted Palindrome (IP) restriction endonucleases cleave symmetrically within their interrupted palindrome binding sites (Kessler et al, 1985; Szybalski et. al, 1991, The Restriction Enzyme Database (REBASE) at http://rebase.neb.com/rebase/). Both IIS and IP restriction endonuclease generate DNA fragments with single-stranded cohesive ends not including the recognition sequence of the enzyme. The cohesive ends then can be any sequence but will be specific for a given fragment.

A universal restriction endonuclease can be constructed using a tailored deoxyoligonucleotide adaptor in conjunction with a Class IIS endonuclease (Szybalski, 1985 and U.S. Pat. No. 4,935,357). The adaptor comprises of a single stranded region complementary to a single stranded region of target DNA at the desired cleavage sites. Adjacent to the single stranded region of the adaptor is a hairpin region containing the recognition sequence of the endonuclease. The adaptor is constructed so that the endonuclease will bind to a recognition sequence in the double stranded portion and will cleave the single stranded target region at the desired site, once the adaptor has been hybridized to the complementary region of the target DNA.

Brenner, S. and Livak, K. J. reported a method of DNA sequencing by stepwise ligation and cleavage (Brenner and Livak, 1989; Brenner, U.S. Pat. Nos. 5,552,278; 5,599,675; 5,856,093; 5,831,065; 5,714,330). In their method, DNA was cleaved by a Type IIS endonuclease to generate fragments with single stranded overhangs having non-identical sequences. DNA polymerase was used to attach fluorescent-labeled nucleotides complementary to the bases in the cohesive ends. Cleavage using a different endonuclease was carried out resulting in the presence of some fragments having fluorescent-labeled ends. Analysis of the fluorescent labeled DNA by gel electrophoresis in an automated DNA sequencing apparatus provided the sequence of the fragment cohesive ends produced by the Type IIS endonuclease cleavage and the length of the fragments.

DNA indexing was developed to define, amplify and isolate specific DNA fragments from complex genomic digests of Type IIS and Interrupted Palindrome (IP) restriction endonucleases (Smith, 1992; Unrau and Deugau, 1994; Deugau et al, U.S. Pat. No. 5,508,169; Cantor, U.S. Pat. No. 5,503,980; Zheleznaya, et al, 1995; Albrecht et al., U.S. Pat. No. 6,013,445; DuBridge et al., U.S. Pat. Nos. 5,888,737; 6,175,002) or Type II restriction endonucleases (Kato U.S. Pat. No. 5,707,807; Sibson & Gibbs, 2001; Guilfoyle et al., 1997; U.S. Pat. Nos. 6,280,948; 6,228,999; 5,994,068). Nucleic acid indexing linkers, containing protruding single strands complementary to the cohesive ends of endonuclease cleavage sites are covalently linked to the DNA fragments through ligation. Selection of the appropriate indexer for each end of a targeted restriction fragment thus can incorporate two common-primer-binding sequences onto the ends of that fragment, allowing specific amplification of the target fragment. DNA indexing provides a non-cloning method to manipulate DNA fragments and separate them into well-characterized subsets. Large number of targets can be amplified simultaneously through this approach. Different from PCR, it doesn't require prior knowledge of the nucleic acid sequences flanking the region of interest, and can be used for characterization of unknown gene region and regions that can't be cloned. Without the cost of two unique primers per target, non-repetitive analyses of large numbers of different targets become feasible.

A limitation of DNA indexing has been its fidelity (Sibson & Gibbs, 2001). Loss of fidelity could occur during ligation of the indexing linkers or purification of the indexed fragments. The fidelity of ligation decreases as the length of the overlapping regions to be joined. However, longer cohesive ends are required to achieve high level of indexing. Another limitation is that it's difficult to separate indexed DNA from the rest of the mixture.

All patents, patent applications, and publications cited herein are hereby incorporated by reference in their entirety. It should be noted that reference to a publication in this Background section is not an admission that the publication constitutes prior art to the instant invention.

### BRIEF SUMMARY OF THE INVENTION

One of the objectives of the present invention is to construct panel of circularization indexers (C-indexers), which will allow circularization and purification of any nucleic acid, sequence or sequences, and allow selective manipulation of the circularized nucleic acid sequence.

Another objective is to provide procedure for using C-indexers to circularize and purify nucleic acid fragments generated by restriction endonuclease digestion.

Another objective is to provide a procedure to circularize and purify nucleic acid fragments from a complex nucleic acid mixture, for identifying, isolating, mapping, amplifying or sequencing said fragments.

Another objective is to provide a kit for generating single stranded circular DNA targets from a complex DNA mixture, for identifying, isolating, mapping, amplifying or sequencing said DNA targets.

Another objective is to provide a kit for generating single stranded circular RNA targets from a complex RNA mixture, for identifying, isolating, mapping, amplifying or sequencing said RNA targets.

Another objective is to provide kits for high-level multiplexing amplification of nucleic acids.

Another objective of the present invention is to construct a nucleic acid structure in which the nucleic acid can be self-primed and amplified without external primers.

Another objective of the present invention is to provide a method of amplifying nucleic acid on solid surfaces without external primers.

Yet another objective of the present invention is to provide kits and procedures of using the kits to amplify nucleic acid in high-multiplex fashion without the need of external primers. The kits can be used to amplify nucleic acids in solution or on solid surfaces.

The present invention combines the techniques used in molecular cloning and DNA indexing to provide flexible and cost effective methods for circularizing and purifying nucleic acids, preferred DNA, from a complex nucleic acid mixture. The circularized DNAs can be amplified in high-level multiplexing fashion without the need for gene-specific primers and can be also be used in a wide range of RCA applications known in art of nucleic acid analysis.

In one aspect, the invention provides a method for indexing double-stranded nucleic acid fragments from a mixture of double-stranded nucleic acid fragments, said method comprises the steps of: (a) treating nucleic acids with one or more restriction endonucleases selected from the group consisting of Type IIS restriction endonuclease and interrupted palindrome (IP) restriction endonuclease to generate fragments having at least one 3′- or 5′-protruding single strand; (b) incubating a C-indexer with said fragments generated in step (a) to allow selective hybridization between complementary protruding single strand of said fragments and said C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of the fragments, wherein said C-indexer is selected from a collection of C-indexers whose 3′- or 5′-protruding single strands collectively encode up to all possible permutations and combinations of nucleotides, A, C, G and T; (c) adding ligase to selectively ligate said C-indexer to those fragments whose 3′- or 5′-protruding single strands are fully complementary to the 3′- or 5′-protruding single strands of said C-indexer; and (d) obtaining circular indexed nucleic acid fragments, wherein the first strand of said C-indexer is ligated to the fragments to form a closed circular strand, and wherein the second strand of said C-indexer is ligated to the fragments to form a discontinuous strand.

The method may further comprises steps of immobilizing said circular indexed nucleic acid fragments to a surface treated with an affinity target capable of binding the affinity tag covalently attached to the second strand of the C-indexer; washing away linear nucleic acid fragments; denaturing the closed circular strand and the discontinuous strand of the circular indexed fragments to release the closed circular strand from the solid surface; and collecting the released closed circular strand of the circular indexed fragments. In some embodiments, the affinity tag is a biotin moiety and the solid surface is streptavidin or avidin coated plate or magnetic beads.

The invention also provides a method of amplifying circular indexed nucleic acid fragments, said method comprises the steps of: (a) treating nucleic acids with one or more restriction endonucleases selected from the group consisting of Type IIS restriction endonuclease and interrupted palindrome (IP) restriction endonuclease to generate fragments having at least one 3′- or 5′-protruding single strand; (b) incubating a C-indexer with said fragments generated in step (a) to allow selective hybridization between complementary protruding single strand of said fragments and said C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of the fragments, wherein said C-indexer is selected from a collection of C-indexers whose 3′- or 5′-protruding single strands collectively encode up to all possible permutations and combinations of nucleotides, A, C, G and T; (c) adding ligase to selectively ligate said C-indexer to those fragments whose 3′- or 5′-protruding single strands are fully complementary to the 3′- or 5′-protruding single strands of said C-indexer; (d) obtaining circular indexed nucleic acid fragments, wherein the first strand of said C-indexer is ligated to the fragments to form a closed circular strand, and wherein the second strand of said C-indexer is ligated to the fragments to form a discontinuous strand; and (e) amplifying the closed circular strand of said circular indexed nucleic acid fragments by rolling circle amplification. In some embodiments, the discontinuous strand is used as rolling circle amplification primer. In some embodiments, the circular indexed nucleic acid fragments are immobilized to a surface through an affinity tag covalently attached to the second strand of said C-indexer before said rolling circle amplification. In some embodiments, the surface is a plate, a chip or a bead. In some embodiments, the rolling circle amplification is performed on the surface. In other embodiments, the rolling circle amplification is performed after said closed circular strand is separated from said discontinuous strand.

The invention also provides a kit for indexing double-stranded nucleic acid fragments from a mixture of double-stranded nucleic acid fragments, said kit comprising: (a) one or more Type IIS or IP restriction endonucleases; (b) a C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of nucleic acid fragments generated by Type IIS or IP restriction endonucleases digestion, wherein said C-indexer is selected from a collection of C-indexers whose 3′- or 5′-protruding single strands collectively encode up to all possible permutations and combinations of nucleotides, A, C, G and T; and (c) ligase and ligase buffer. The kit may further comprises a nucleic acid polymerase (e.g., a DNA polymerase), a mixture of nucleotide precursors (e.g., all four deoxynucleotide precursors), and a rolling circle amplification buffer.

For the methods and kits used for indexing double-stranded nucleic acid described herein, the lengths of the 3′- or 5′-protruding single strand on the first strand and the second strand of the C-indexer may be the same or different. The length of the 3′- or 5′-protruding single strand of the C-indexer may be any length longer than 2 bases, preferably 3, 4, or 5 bases. The restriction endonuclease may be a Type IIS restriction endonuclease (e.g., FokI, AarI, and HgaI) or interrupted palindrome (IP) restriction endonuclease. In some embodiments, the second strand of the C-indexer contains at least one nick, wherein the nick cannot be ligated. In some embodiments, the 5′ end of the second strand of the C-indexer cannot be ligated (for example, the 5′ end of the second strand is not phosphorylated. In some embodiments, the second strand of the C-indexer contains at least one gap or overlap. In some embodiments, the second strand includes an affinity tag such as a biotin moiety, a digoxigenin (DIG), a reactive amine, thiol group, an antibody, an antigen or oligonucleotide covalently attached to the second strand of said C-indexer. The affinity tag may be at the 5′ end of the second strand. The affinity tag may be at an internal base of the second strand. In some embodiments, the double-stranded nucleic acid fragments indexed are genomic DNA or cDNA.

In another aspect, the invention provides a method for indexing single-stranded nucleic acid fragments from a mixture of single-stranded nucleic acid fragments, said method comprises the steps of: (a) incubating a C-indexer with single-stranded nucleic acid fragments to allow selective hybridization between complementary protruding single strand of said C-indexer with the 5′ end and 3′ end of said single-stranded nucleic acid fragments; wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; (b) adding ligase to selectively ligate the first strand of said C-indexer with those single-stranded nucleic acid fragments whose 5′- and 3′-ends are fully complementary, respectively, to the 5′- and 3′-protruding single strands of said C-indexers; and (c) obtaining circular indexed nucleic acid fragments, wherein the first strand of said C-indexer is ligated to the nucleic acid fragment to form a closed circular strand, wherein the second strand of the C-indexer is not closed.

The invention also provides a method of amplifying circular indexed single-stranded nucleic acid fragments, said method comprises the steps of: (a) incubating a C-indexer with single-stranded nucleic acid fragments to allow selective hybridization between complementary protruding single strand of said C-indexer with the 5′ end and 3′ end of said single-stranded nucleic acid fragments; wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; (b) adding ligase to selectively ligate the first strand of said C-indexer with those single-stranded nucleic acid fragments whose 5′- and 3′-ends are fully complementary, respectively, to the 5′- and 3′-protruding single strands of said C-indexer; (c) obtaining circular indexed nucleic acid fragments, wherein the first strand of said C-indexer is ligated to the nucleic acid fragment to form a closed circular strand, wherein the second strand of said C-indexer is not closed; and (d) amplifying the closed circular strand of said circular indexed nucleic acid fragments by rolling circle amplification. In some embodiments, the circular indexed nucleic acid fragments are immobilized to a surface treated with an affinity target capable of binding to the affinity tag covalently attached to the second strand of said C-indexer before said rolling circle amplification. In some embodiments, the affinity tag is a biotin moiety, a digoxigenin (DIG), a reactive amine, thiol group, an antibody, an antigen or oligonucleotide. In some embodiments, the surface is a plate, a chip or a bead coated with affinity target capable of binding to the affinity tag. In some embodiments, the rolling circle amplification is performed on the surface. In some embodiments, the rolling circle amplification is performed after said closed circular strand is separated from said discontinuous strand. In some embodiments, the single-stranded nucleic acids can be generated from denaturing double-stranded nucleic acids.

The invention also provides a kit for indexing single-stranded nucleic acid fragments from a mixture of single-stranded nucleic acid fragments, said kit comprising: (a) a C-indexer, wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; and (b) ligase and ligase buffer. The kit may further comprise a nucleic acid polymerase (such as DNA polymerase and RNA polymerase), a mixture of nucleotide precursors (such as deoxynucleotide precursors, ribonucleotide precursors, and modified nucleotide precursors), and a buffer for rolling circle amplification reaction. The kit may also further comprise a solid surface (such as streptavidin coated plate or magnetic beads).

For the methods and kits for indexing single-stranded nucleic acid described herein, the nucleic acid may be DNA, RNA, synthetic oligonucleotides, or any other configurations. The 5′- and 3′-protruding single strand on the second strand of the C-indexer may be any lengths and sequences. The lengths of the 5′- and 3′-protruding single strand on the second strand of the C-indexer may be the same or different. In some embodiments, the lengths of the 5′- and 3′-protruding single strand on the second strand of the C-indexer are between 3 and 30. In some embodiments, the second strand of the C-indexer contains at least one nick, wherein the nick cannot be ligated. In some embodiments, the 5′ end of the second strand of the C-indexer cannot be ligated (for example, the 5′ end of the second strand is not phosphorylated). In some embodiments, the second strand includes an affinity tag (such as a biotin moiety a digoxigenin (DIG), a reactive amine, thiol group, an antibody, an antigen or oligonucleotide covalently attached to the second strand of the C-indexer. In some embodiments, the affinity tag is at the 5′ end of the second strand. In some embodiments, the affinity tag is at an internal base of the second strand. In some embodiments, the single-stranded nucleic acids can be generated from denaturing double-stranded nucleic acids.

In some embodiments of methods and kits described herein, two or more different C-indexers may be used to index two or more subsets of nucleic acid fragments.

The invention also provides C-indexers, such as C-indexers described in Examples 1-3.

### BRIEF DESCRIPTION OF THE SEVERAL VIEWS OF THE DRAWING(S)

FIG. 1 is a graph showing constructing C-indexers by mixing sense and antisense strands. Different combinations of sense and antisense strands yield different C-indexers. The solid line and dashed line stand for the fixed sequence region of sense and antisense strand respectively, and N, M, X and Y stands for variable bases.

FIG. 2 is a graph showing a process of C-indexing a double-stranded DNA.

FIG. 3 is a graph showing purification of a single-stranded circular indexed DNA.

FIG. 4 is a graph showing modified RCA process.

FIG. 5 is a graph showing self-assembled C-indexed nucleic acid amplification.

FIG. 6 is a graph showing self-assembled C-indexed nucleic acid amplification on a solid surface.

FIG. 7 is a graph showing self-assembled amplification of C-indexed single-stranded nucleic acid.

FIG. 8 is a graph showing an example of C-indexer DNA detection without DNA amplification.

FIG. 9 is a graph showing an example of C-indexer DNA detection and genotyping without DNA amplification.

### DETAILED DESCRIPTION OF THE INVENTION

The present invention describes methods to index nucleic acid using both ends of a nucleic acid fragment simultaneously and provides much higher indexing specificity. The method is capable of indexing at various specificity levels including indexing a specific nucleic acid sequence from a complex nucleic acid mixture (such as human genomic DNA restriction digest). Both double-stranded and single-stranded nucleic acids including RNAs, and synthetic oligonucleotides can be indexed with the present invention. The present invention puts indexed nucleic acid in a closed circle so that they can be amplified by any nucleic acid amplification or replication techniques including the rolling circle amplification (RCA). The circular indexed nucleic acids can be amplified in a very high level multiplexing fashion without requiring external primers. The amplified nucleic acids can be used in any applications such as hybridization, nucleic acid detection, and sequencing. Function modifications such as restriction enzyme recognition sits can be encoded into the C-indexers to facilitate down stream applications. Depends on the down stream application, the amplified nucleic acid can be either single-stranded or double-stranded. The present invention also provides a method to selectively enrich and purify nucleic acids before amplification so that nucleic acid can be detected at very low level. The present invention also provides methods for making high density nucleic acid arrays in which each immobilized single-stranded nucleic acid contains thousand copies of the tandem repeated sequences. Such arrays have higher nucleic acid concentration for a given surface area and will be able to capture target more efficiently. A high density cDNA array can be made by index total cDNA library into subsets and immobilize each subset at a different location on array. This cDNA array can be used for whole genome expression profiling, gene discovery, drug discovery and screening.

The practice of the present invention will employ, unless otherwise indicated, conventional techniques of immunology, molecular biology, microbiology, cell biology and recombinant DNA, which are within the skill of the art. See, e.g., Molecular Cloning: a laboratory manual, 2^{nd} edition Sambrook, et al. (1989); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

For clarity of disclosure, and not by way of limitation, the detailed description of the invention is divided into the subsections that follow. Before the subject invention is further described, it is to be understood that the invention is not limited to the particular embodiments of the invention described below, as variations of the particular embodiments may be made and still fall within the scope of the appended claims.

I. Definitions

Unless defined otherwise, all technical and scientific terms used herein have the same meaning as is commonly understood by one of ordinary skill in the art to which this invention belongs. All patents, applications, published applications referred to herein are incorporated by reference in their entirety. If a definition set forth in this section is contrary to or otherwise inconsistent with a definition set forth in applications, published applications that are herein incorporated by reference, the definition set forth in this section prevails over the definition that is incorporated herein by reference.

As used herein, “a” or “an” means “at least one” or “one or more.”

As used herein, “nucleic acid (s)” refers to any sized multimer of nucleotide monomeric units in any form, including inter alia, single-stranded, duplex, triplex, linear and circular forms. It includes short multimers such as dimers, trimers, and the like. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

“Solid surface” can include any solid material to which affinity targets can be coupled. This includes materials such as acrylamide, cellulose, nitrocellulose or materials comprising plastic, silicon, metal, and glass. Solid surface can have any useful form including thin films or membranes, beads, bottles, dishes, fibers, woven fibers, shaped polymers, particles and microparticles plates, or slides. A preferred form for a solid surface is a bead, a plate or a slide.

II. Indexing Nucleic Acids by Circularization Indexers (C-Indexer)

The invention provides a method for indexing double-stranded nucleic acid fragments from a mixture of double-stranded nucleic acid fragments, said method comprises the steps of: (a) treating nucleic acids with one or more restriction endonucleases selected from the group consisting of Type IIS restriction endonuclease and interrupted palindrome (IP) restriction endonuclease to generate fragments having at least one 3′- or 5′-protruding single strand; (b) incubating a C-indexer with said fragments generated in step (a) to allow selective hybridization between complementary protruding single strands of said fragments and said C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of the fragments, wherein said C-indexer is selected from a collection of C-indexers whose 3′- or 5′-protruding single strands collectively encode up to all possible permutations and combinations of nucleotides, A, C, G and T; (c) adding ligase to selectively ligate said C-indexer to those fragments whose 3′- or 5′-protruding single strands are fully complementary to the 3′- or 5′-protruding protruding single strands of said C-indexer; and (d) obtaining circular indexed nucleic acid fragments, wherein the first strand of said C-indexer is ligated to the fragments to form a closed circular strand, and wherein the second strand of said C-indexer is ligated to the fragments to form a discontinuous strand.

The invention also provides a method for indexing single-stranded nucleic acid fragments from a mixture of single-stranded nucleic acid fragments, said method comprises the steps of: (a) incubating a C-indexer with single-stranded nucleic acid fragments to allow selective hybridization between complementary protruding single strand of said C-indexer with the 5′ end and 3′ end of said single-stranded nucleic acid fragments; wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; (b) adding ligase to selectively ligate the first strand of said C-indexer with those single-stranded nucleic acid fragments whose 5′- and 3′-ends are fully complementary, respectively, to the 5′- and 3′-protruding single strands of said C-indexers; and (c) obtaining circular indexed nucleic acid fragments, wherein the first strand of said C-indexer is ligated to the fragments to form a closed circular strand, wherein the second strand of the C-indexer not closed.

1. Construction of C-Indexers

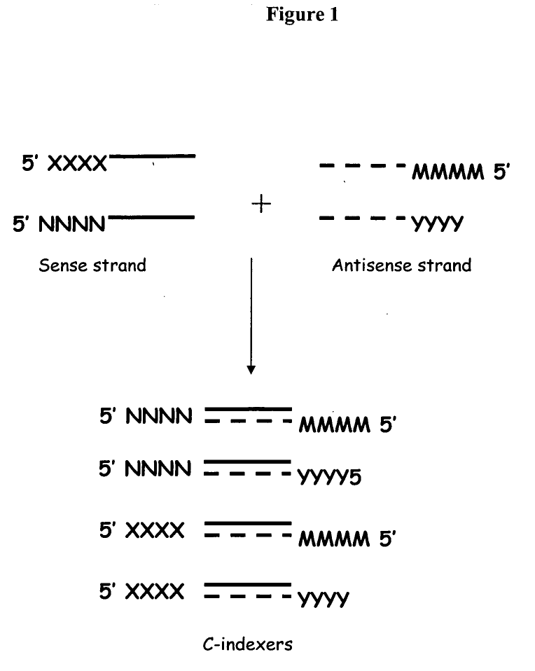

The present invention describes methods to construct circularization indexers (C-indexer) and use them to circularize nucleic acid, preferably deoxyribonucleic acid (DNA), released from endonuclease digestions. For indexing double-stranded nucleic acid fragments, the C-indexers of the invention are double stranded (ds) polynucleotides that have two unique cohesive ends containing any number of bases, preferably 3, 4, 5 bases (FIG. 1). The C-indexer can be made from two separate single-stranded (ss) polynucleotides defined as sense-strand (interchangeably termed “the first strand” herein) and antisense-strand (interchangeably termed “the second strand” herein) respectively.

The sense-strand sequences can be constructed by adding variable sequence of n bases to the 3′- or 5′-end of a fixed sequence A. A set of sense-strand contains sequences of the same length of variable sequence. The variable sequences of members of said set of sense-strand encode collectively up to all possible permutations and combinations of nucleotides, A, C, G and T. Because there are 4ⁿ possible sequence combinations for n bases, there are 4ⁿ sequences in a set of sense-strand. For example, there are 256 sense-strand sequences for C-indexers with four-base cohesive ends. Similarly, a set of antisense-strand sequences can be constructed by adding variable sequence of m bases to the 3′- or 5′-end of sequence B. A set of antisense-strand also include variable sequences that encode up to all possible permutations and combinations of nucleotides, A, C, G and T. For C-indexers with four-base cohesive ends, there are 256 sequences in its antisense-strand set.

Mixing sequences from sense-strand and antisense-strand sets produces a set of C-indexer sequences. The cohesive ends of C-indexers in a set of C-indexers collectively encode up to all possible permutations and combinations of nucleotides, A, C, G and T. If there are N sequences in sense-strand and antisense-strand set respectively, combination of the two sets will produce N*(N+1)/2 possible duplex sequences with identifiable cohesive ends. For example, 256 sense-strand sequences and 256 antisense-strand sequences with four-base variable end can generate up to 32,896 C-indexers with unique four-bases cohesive ends. The two cohesive ends in a C-indexer can either be the same length or different lengths.

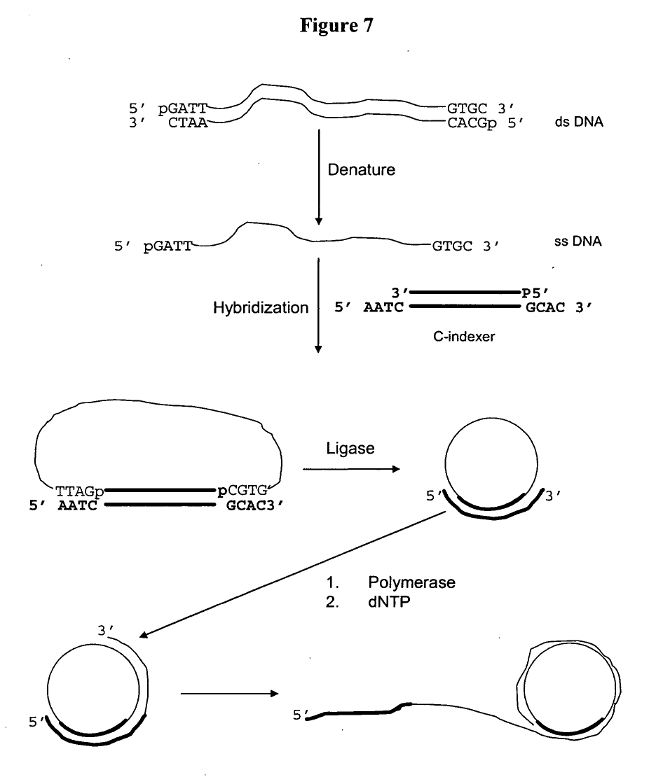

C-indexers can also be used to circularize single-stranded nucleic acid. A C-indexer for circularizing single-stranded nucleic acid has both 5′ and 3′ cohesive ends on the antisense strand or the second strand (therefore one strand is longer than the other strand as show in FIG. 7). 5′ end of the shorter strand (interchangeably termed “the first strand” herein) is phosphorylated and sequences of the 5′ and 3′ cohesive ends are complementary to the 5′ and 3′ end of the nucleic acid target respectively. After ligation, the single-stranded nucleic acid can be circularized with the shorter stranded of the C-indexer. The longer strand (interchangeably termed “the second strand” herein) of the C-indexer can be hybridized to the single-stranded circular template containing the target.

The circular indexed nucleic acids can be amplified by any nucleic acid amplification techniques, preferably by rolling circle amplification (RCA). The C-indexers that can be used for rolling circle amplification are a linear double-stranded nucleic acid molecule where one of the strands will become part of the continuous strand of the circular indexed nucleic acid and the other strand will become part of the discontinuous strand of the circular indexed nucleic acid. For identification, the strand of the C-indexer which will become part of the continuous strand of the circular indexed nucleic acid is referred to as the first strand of the C-indexer, and the strand of the C-indexer which will become part of the discontinuous strand of the circular indexed nucleic acid is referred to as the second strand of the C-indexer.

All of the ends present in the first strand of the C-indexer, including internal ends, if present, should be ligatable. Ligatable ends are ends that can be ligated to compatible ends by ligase, or which can otherwise be coupled to compatible ends. Preferred ligatable ends are nucleotides having a 3′ hydroxyl or a 5′ phosphate. Internal ends are ligatable only if compatible ends are adjacent. For example, a nick with a 3′ hydroxyl-on one end and a 5′ phosphate on the other end is a ligatable nick and the ends are ligatable. Nick has its usual meaning. Specifically, a nick is a break in a strand hybridized to another strand where there are no unpaired nucleotides in the other strand opposite the nick.

To result in a discontinuous second strand in the circular indexed nucleic acid, the second strand of the C-indexer may contain at least one non-ligatable end or at least one gap or overlap. Non-ligatable ends are ends that cannot be ligated to compatible ends by ligase, or which cannot otherwise be coupled to compatible ends. Preferred non-ligatable ends are nucleotides having a blocking group at the 3′ or 5′ position. For example, the second strand of the C-indexer can include a 3′-terminal or 5′-terminal biotin residue (either at the end of a continuous second strand or at a nick in a discontinuous second strand). This residue renders the terminus non-ligatable, causing all circular indexed nucleic acids to contain a nick after circularizing target nucleic acids by ligation. This biotin residue can then used as a handle to remove the second strand of the circular indexed nucleic acid, generating single-stranded circles for amplification. Thus, the biotin is both a blocking group and an affinity tag.

Internal ends are also non-ligatable if, for example, compatible ends are not adjacent. For example, a nick with a 3′ hydroxyl on both ends is an unligatable nick and the ends are unligatable. A nick with a blocking group on one of the ends is also an unligatable nick and the end with the blocking group is an unligatable end. Both gaps and overlaps is not ligatable even if the ends would otherwise be compatible since the ends are not close enough to be coupled. Gap has its usual meaning. Specifically, a gap is a break in a strand hybridized to another strand where there is at least one nucleotide on the other strand opposite the gap that is unpaired. A gap can also occur at the end of the C-indexer in that a nucleic acid molecule, when hybridized to sticky ends of the C-indexer, can fail to extend to the end of one of the strands of the C-indexer.

An overlap occurs where the adjacent ends of two strands hybridized adjacent to each other on another strand extend beyond the region of hybridization. A preferred form of overlap is where the two overlapping strands hybridize to each other in the overlapping region. This type of overlap in a C-indexer produces a Y shaped molecule such as molecules illustrated in U.S. Pat. No. 6,287,824.

The second strand of the C-indexer may contain multiple nicks, gaps, and overlaps in any combination. Any number of such nicks may be ligatable or non-ligatable. All that is required is at least one feature that prevents the second strand of the circular indexed nucleic acid from being continuous following insertion of the nucleic acid molecule. For this purpose, a single non-ligatable end or other non-ligatable feature is all that is required. The first strand of the C-indexer contain multiple nicks so long as they are all ligatable; that is, so long as the first strand of the circular indexed nucleic acid will be continuous following insertion of the nucleic acid molecule.

The second strand of the C-indexer can also contain one or more affinity tags to immobilize the circular indexed nucleic acids and to facilitate separation of the first and second strands of the circular indexed nucleic acid. Affinity tags can be attached to the second strand of the C-indexer at the 5′ end or at an internal base.

An “affinity tag” is a molecule that interacts specifically with a particular molecule or moiety. The molecule or moiety that interacts specifically with an affinity tag is referred to herein as an “affinity target”. Together, an affinity tag and affinity target make up a binding pair. Either member of a binding pair can be used as an affinity tag and either member can be used as an affinity target. An affinity tag is the member of the binding pair coupled to the C-indexer. It is to be understood that the term affinity target refers to both separate molecules and to portions of molecules, such as an epitope of a protein, that interacts specifically with an affinity tag. Antibodies, either member of a receptor/ligand pair, and other molecules with specific binding affinities are examples of affinity tags, useful as the affinity portion of a reporter binding molecule. By coupling an affinity tag to the second strand of a C-indexer, binding of the affinity tag to its affinity target allows immobilization of the circular indexed nucleic acid and the separation of the first and second strands of the circular indexed nucleic acid. An affinity tag that interacts specifically with a particular affinity target is said to be specific for that affinity target. For example, an affinity tag which is an antibody that binds to a particular antigen is said to be specific for that antigen. The antigen is the affinity target. Complementary nucleotide sequences can be used as binding pairs. Exemplary affinity tags or affinity targets are haptens such as biotin, digoxigenin (DIG) and their antibodies, reactive amine, thiol group, antibody, antigen, oligonucleotide, streptavidin, avidin, phenylene diisothiocyanate, disuccinimidylcarbonate, disuccinimidyloxolate, or dimethylsuberimidate.

It is preferred that the ends of the C-indexer, when ready for ligation, do not contain compatible ends that can be ligated. This will prevent the circularization of C-indexers in the absence of insertion of a nucleic acid molecule.

In the present invention, the fixed sequence region of the said C-indexer can be any length, any sequence and may encode any functional modification known in the art of nucleic acid manipulation. Upon circularization, the functional modification encoded in C-indexer is attached to the circularized nucleic acid and enable flexible manipulations of the circularized sequences. Examples of using these functional modifications are demonstrated in the following embodiments. Other functional modifications known in the art can be used.

In a preferred embodiment, universal primer sequences are encoded into the fixed sequence region of the C-indexer. This allows high-level multiplexing amplification of the circularized DNA fragments with the same universal primer sequences.

In another preferred embodiment, an affinity ligand (used interchangeably with the term “tag”), e.g. a biotin moiety, is covalently linked to the fixed sequence region of the C-indexer. The affinity tag will enable immobilization of the circularized DNA fragments.

In another embodiment, a restriction enzyme recognition sequence is encoded in the fixed sequence region of the C-indexer.

In yet another preferred embodiment, the fixed sequence region of the C-indexer encodes single stranded sequence of the oligonucleotide adaptor of universal endonuclease (Szybalski, 1985). The circularized DNA can then be manipulated using universal endonuclease.

The sense-strand and antisense-strand polynucleotides of the C-indexer may be prepared by methods known in the art. A preferred method is the chemical synthesis using phosphoramidite chemistry. Members of the sense-strand and antisense-strand sets are synthesized and stored separately on the basis of the identity of their 3′- or 5′-cohesive end. Mixing different sense-strand and antisense-strand polynucleotides yields a set of C-indexers. Members of the C-indexer set are physically separated from each other on the basis of the identities of their two 3′- or 5′-cohesive ends. The same set of C-indexers can be used in conjunction with any Type IIS or IP restriction endonuclease that generate a length of 3′- or 5′-cohesive ends at cleavage site corresponding to the length of the 3′- or 5′-cohesive ends of the C-indexers.

The invention provides a method of constructing circularization indexers (C-indexers), comprising the steps of: (a) constructing a set of sense-strand sequences by adding variable sequence of n bases to either 3′- or 5′-end of a fixed polynucleotide sequence A; (b) constructing a set of antisense-strand sequences by adding variable sequence of m bases to either 3′- or 5′-end of a fixed polynucleotide sequence B; and (c) mixing sequences from said sense-strand set with sequences from said antisense-strand set to generate a set of C-indexers; wherein said C-indexer comprises a double stranded fixed sequence region and two 3′- or 5′-single stranded protruding ends.

n and m can be any number. In some embodiments, n and/or m are 3, 4, and 5. In some embodiments, n and m are the same number. In some embodiments, n and m are different number.

In some embodiments, the variable sequence includes sequences that encode up to all possible permutations and combinations of nucleotides, A, C, G and T. In some embodiments, the set of C-indexers contains sequences whose 3′- or 5′-cohesive ends collectively encode up to all possible permutations and combinations of nucleotides, A, C, G and T. In some embodiments, the fixed polynucleotide sequences A and B are fully complementary to each other and can be any length and any sequence.

In some embodiments, the double stranded sequence formed by sequence A and B contains functional modifications that can be used to manipulate indexed nucleic acid. In some embodiments, the functional modification is primer sequence that will initiate a polymerase reaction. In some embodiments, the functional modification can be a covalently attached affinity tag, such as biotin. In some embodiments, the functional modification is the single stranded sequence of a universal endonuclease oligonucleotide adaptor. In some embodiments, the functional modification is restriction recognition sequence.

In some embodiments, the sense and antisense strands are any polynucleotide. In some embodiments, the polynucleotide is a DNA oligonucleotide chemically synthesized using phosphoramidite chemistry.

2. Circularization and Purification of DNA Fragments

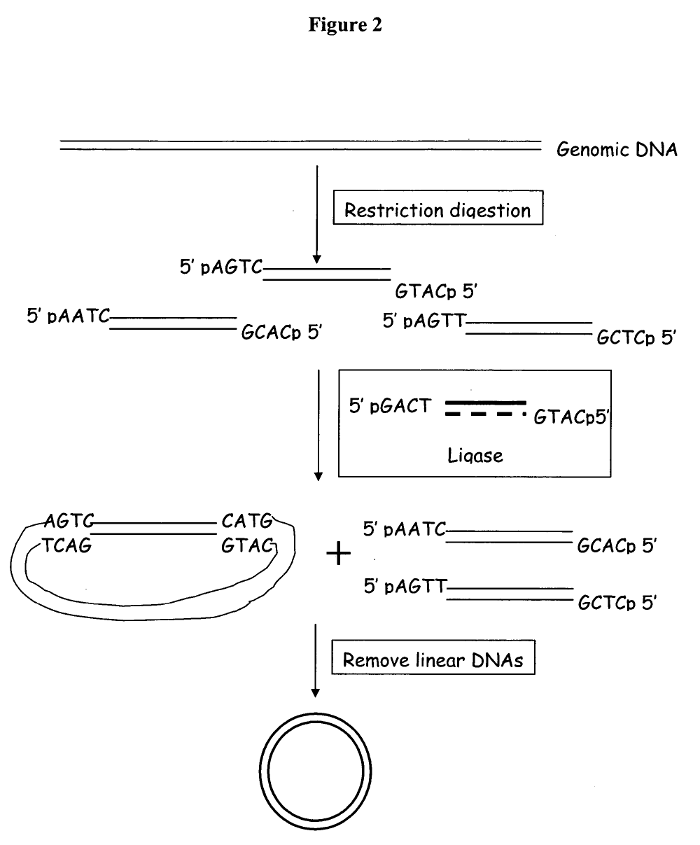

Molecular cloning is a well-known technique in the art of molecular biology. One of the steps in this procedure is to insert DNA fragments released from restriction endonuclease digestion into double stranded plasmids (Cohen et al., 1973; Tanaka and Weisblum, 1975) whose cohesive ends are complementary to the cohesive ends of the fragments. DNA Ligase covalently links the fragments to the plasmids to form double stranded circular DNAs. In the present invention, nucleic acid fragments, preferably DNA, released from a restriction endonuclease digestion are circularized in a similar manner except the plasmids are replaced by C-indexers. DNA fragments are circularized through ligation to those C-indexers whose cohesive ends are complementary to the cohesive ends of the fragments (FIG. 2 for circularizing double-stranded nucleic acid fragments and FIG. 7 for circularizing single-stranded nucleic acid fragments). Ligation is carried out at sufficiently low DNA concentration so that intermolecular ligation between fragments or C-indexers is negligible. Self-circularization could occur for those fragments whose two cohesive ends are complementary to each other, but can be minimized by using excess amount of C-indexers.

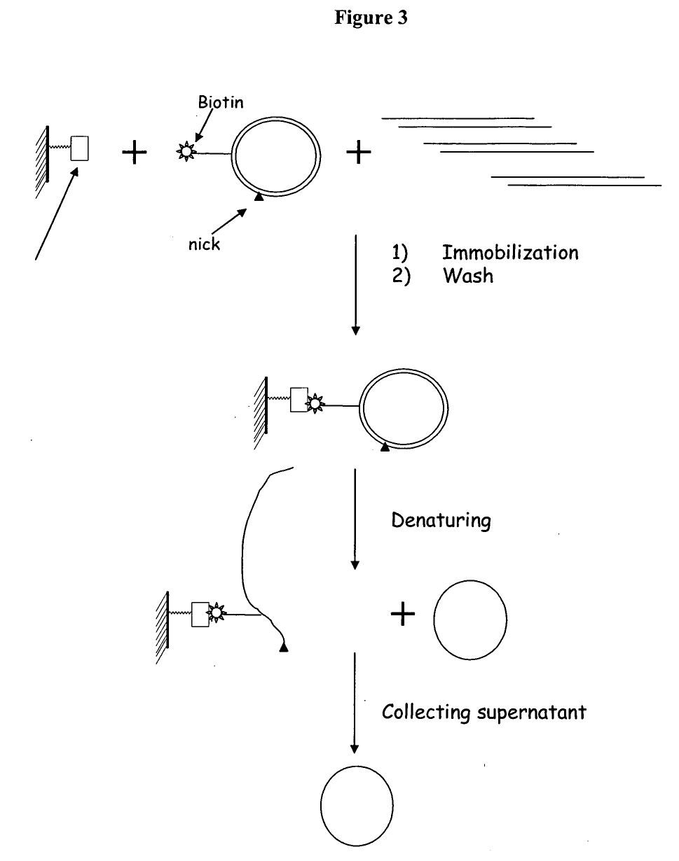

Due to the significant structural differences, the circularized DNA can be easily separated from the linear DNA by methods known in the art. In a preferred embodiment, circularized DNA bounds to streptavidin magnetic bead or surface through a biotin linked to an internal base of the C-indexer. The C-indexer strand containing the internal linker does not have a 5′ phosphate and the circularized DNA, therefore, has a nick on this strand. Under denaturing condition, the immobilized strand will be linearized and facilitate the release of single-strand circular DNA (FIG. 3). In another preferred embodiment, linear DNAs are removed by exonuclease treatment.

In the present invention, circular indexed nucleic acids can be immobilized to a solid surface include any solid material to which affinity targets can be coupled. This includes materials such as acrylamide, cellulose, nitrocellulose or materials comprising plastic, silicon, metal, and glass. Solid surface can have any useful form including thin films or membranes, beads, bottles, dishes, fibers, woven fibers, shaped polymers, particles and microparticles plates, or slides. A preferred form for a solid surface is a bead, a plate or a slide.

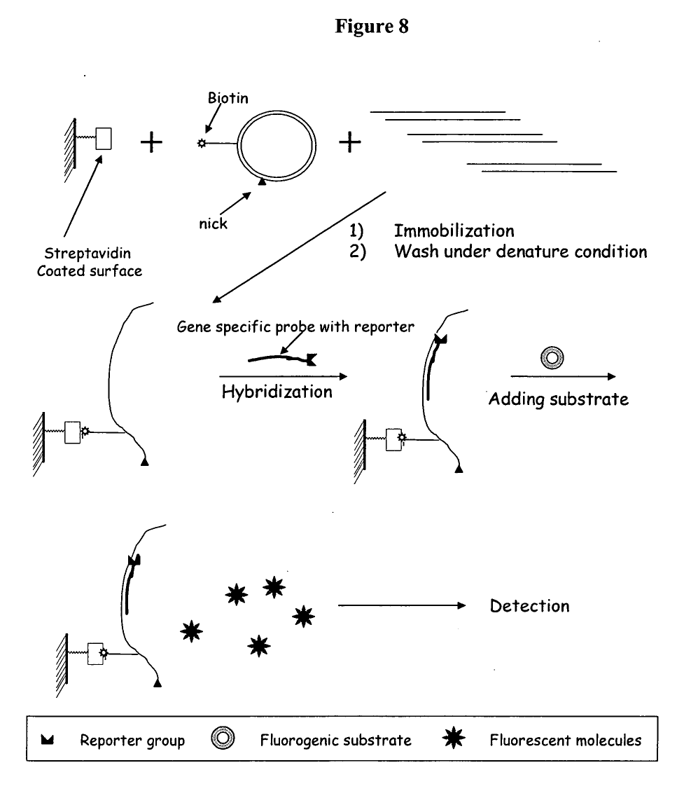

C-indexer circularization and purification method described in the invention can be adapted for any nucleic acid (e.g., DNA and RNA) such as genomic DNA, cDNA (cloned copies of messenger ribonucleic acid), mtDNA (mitochondria DNA), mRNA, rRNA and from any sources such as human, animals, plants, pathogens and other biological agents. The process described above can be used to enrich the circularized DNA fragments, which can then be detected without amplification (FIG. 8).

In some embodiments, the invention provides a method for indexing and obtaining circularized DNA targets from a complex DNA mixture, comprising the steps of: (a) treating nucleic acids, preferably DNA, with one or more restriction endonuclease to generate fragments having at least one 3′ or 5′ n-base protruding single strand (cohesive end); (b) mixing said C-indexers with said DNA fragment, wherein at sufficiently low DNA concentration and the presence of excessive amount of C-indexers, the cohesive ends of said C-indexers hybridize to the complementary cohesive ends of the DNA fragments to form circular structures; (c) adding DNA Ligase to the said circular structures to produce double stranded circular DNAs; (d) separating the circularized DNA fragments from linear DNAs and C-indexers; and (e) collecting the circularized DNA fragments. The restriction endonucleases can be Type IIS restriction endonuclease (such as FokI, AarI, or HgaI) or interrupted palindrome (IP) restriction endonucleases. n can be any number, such as 3, 4, and 5. The method may comprise further steps (to purify the circularized DNAs and release the single stranded circular DNAs): (a) immobilizing said double-stranded circular DNAs to a solid surface through an internal biotin covalently attached to said C-indexers; (b) washing away the linear DNAs; and (c) denaturing the double-stranded circular DNAs and collecting single-stranded circular DNAs in the supernatant. In some embodiments, the internal linker is a biotin moiety, and the solid surface is streptavidin coated magnetic beads. In some embodiments, the C-indexer is not phosphorylated at 5′ end on the strand that is covalently attached to the biotin.

Any nucleic acid ligase is suitable for use in the methods described herein. Preferred ligases are those that catalyze the formation of a phosphodiester bond between juxtaposed 5′ phosphate and 3′ hydroxyl termini in duplex DNA or RNA. That is, ligases that fail to ligate the free ends of single-stranded DNA at a significant rate are preferred. Thermostable ligases are especially preferred. Many suitable ligases are known, such as T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE.RTM. (Kalin et al., Mutat Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjamardottir et al., Gene 151:177-180 (1995)). T4 DNA ligase is preferred for ligations involving RNA target sequences due to its ability to join blunt end and cohesive end termini as well as repair single stranded nicks in duplex DNA, RNA, or DNA/RNA hybrids (Engler, M. J. and Richardson, C. C. (1982). In P. D. Boyer (Ed.), The Enzymes Vol. 5, (p. 3). San Diego: Academic Press.)

3. Circularization Indexing (C-Indexing)

The C-indexers of the invention can be used in conjunction with Type IIS or interrupted palindrome (IP) restriction endonucleases to selectively circularize a specific collection of restriction endonuclease digested nucleic acid fragments from a complex nucleic acid mixture or to categorize a complex mixture into subsets with reduced complexity. This process is defined as circularization indexing (C-indexing) (such as shown in FIG. 2 and FIG. 7).

Type IIS restriction endonucleases recognizes asymmetric base sequences and cleave DNA at a specified position outside of the recognition site (Kessler et al, 1985; Szybalski et. al, 1991, The Restriction Enzyme Database (REBASE) at http://rebase.neb.com/rebase/), and interrupted palindrome (IP) restriction endonucleases (Berger SL Anal Biochem. 1994 October;222(1): 1-8) recognizes interrupted palindromic sequences and cleave DNA symmetrically within the interrupted palindromic sequences. Both Type IIS and IP restriction endonuclease cut nucleic acid, preferably DNA, and generate fragments with cohesive ends not including the recognition sequence. The cohesive ends then can be any sequence but will be specific for a given fragment. Depending on the enzyme, number of base in the cohesive ends ranges from 1 to 6 bases, preferably 3,4,5 bases. Examples Type IIS restriction endonucleases are FokI, AarI, HgaI, BbvI, BspMI, SfaNI and more listed by Szybalski et. al (1991) and by Rich Roberts and Dana Macelis (The Restriction Enzyme Database (REBASE) at http://rebase.neb.com/rebase/). Examples of IP restriction endonucleases are SfiI, BglI, and BstXI. and more listed by Rich Roberts and Dana Macelis (The Restriction Enzyme Database (REBASE) at http://rebase.neb.com/rebase/).

The number of bases in the cohesive ends determines the number of possible sequences encoded by the cohesive ends. For example, the number of possible sequences N is 256 (4⁴) for 4-base cohesive ends. Since each DNA fragment has two cohesive ends, there are N*(N+1)/2 classes of fragments that are identifiable based on sequences of cohesive ends. On average, n-base recognition site occurs every 4ⁿ bases. Human genomic DNA has a haploid number of 3×10⁹ base pairs (bp). Complete digestion of human genomic DNA by 5-base endonuclease such as FokI yields approximately 3×10⁶ fragments (3×10⁹/4⁵) with the length around 1024 bp (45). Since FokI generates fragments of 4-base cohesive ends, the fragments can be categorized into 32,896 (256*(256+1)/2) identifiable classes based on sequences of the cohesive ends. Each of these identifiable classes will contain on average 3×10⁶/32,896 fragments, that is about 100 fragments. In another word, a single C-indexer with 4-base protruding single strand on average will index 100 DNA fragments of approximately 1024 bp long from a complete FokI digestion of human genomic DNA. These 100 indexed DNA fragments can be easily purified from the rest of the DNA mixture and then further manipulated or analyzed without the interference of the un-indexed DNAs.

The number of bases in the endonuclease recognition site and the number of bases in the cohesive end at the endonuclease cleavage site determine the indexing specificity and the length of the fragments. In general, the more bases in the recognition site, the longer the fragments. The more bases in the cohesive ends, the higher the indexing specificity. The level of indexing can be adjusted by varying the number of base in the restriction recognition sites and the number of base in the protruding single strand the enzyme generates. For example, one 4 base C-indexer can, on average, index 100 fragments from a complete FokI (a 5 base cutter) digest of human genomic DNA or 20 fragments from a complete BsaI (a 6 base cutter) digest of human genomic DNA. There are a large number of Type IIS and IP endonucleases with recognition site ranges from 4-7 bp and cohesive ends range from 1-6 bases long. This provides a great flexibility for choosing the desirable fragments.

With the completion of genome sequencing for human and other organisms (Lander et al., 2001; Venter et al., 2001; GenBank database), it's possible to create a fragment database of different DNAs digested by different endonuclease or mixture of endonuclease. The large number of available Type IIS and IP endonucleases create a giant pool of fragments, from which a computer program can be used to choose endonuclease that will provide the optimal indexing specificity and fragment length for the specific applications. For example, a specific sequence may be found in a BlvI fragment that will be indexed among 200 fragments. The same sequence may also be found in a FokI fragment that will be indexed among 20 fragments. FokI will be the choice for indexing this sequence. Once the fragments and endonuclease are chosen, the C-indexers can be selected to circularize and purify the desired DNA fragments according the procedures described herein. For indexing a large panel of specific sequences, it's also possible to run several separate digestions and circularization and then combine the circularized DNAs for downstream manipulations.

Compared with the reported DNA indexing methods (Smith, 1992; Unrau and Deugau, 1994; Deugau et al, U.S. Pat. No. 5,508,169; Cantor, U.S. Pat. No. 5,503,980; Zheleznaya, et al., 1995; Albrecht et al., U.S. Pat. No. 6,013,445; DuBridge et al., U.S. Pat. No. 5,888,737; 6,175,002), the C-indexing described in the present invention indexes both ends of the fragment with one indexer. This provides higher indexing specificity and fidelity. Another advantage is that the indexed DNA fragments are circularized and can be easily separated from the un-indexed linear fragments. Circular DNA targets also provide flexibility for the downstream manipulations. For example, DNA targets can be amplified in high-level multiplexing fashion without gene-specific primers either by PCR or RCA using primers encoded in the C-indexer. DNA target can also be amplified by RCA using the second strand (discontinuous strand) of the circular indexed nucleic acid to generate single-stranded or double-stranded amplicons depending on the down stream applications.

III. Amplification and Detection and of Indexed Nucleic Acids

The invention also provides methods for amplifying and detecting of indexed nucleic acids. The invention provides a method of amplifying and detecting double-stranded nucleic acid fragments, said method comprises the steps of: (a) treating nucleic acids with one or more restriction endonucleases selected from the group consisting of Type IIS restriction endonuclease and interrupted palindrome (IP) restriction endonuclease to generate fragments having at least one 3′- or 5′-protruding single strand; (b) incubating a C-indexer with said fragments generated in step (a) to allow selective hybridization between complementary protruding single strand of said fragments and said C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of the fragments, wherein said C-indexer is selected from a collection of C-indexers whose 3′- or 5′-protruding single strands collectively encode up to all possible permutations and combinations of nucleotides, A, C, G and T; (b) adding ligase to selectively ligate said C-indexer to those fragments whose 3′- or 5′-protruding single strands are fully complementary to the 3′- or 5′-protruding protruding single strands of said C-indexer; (c) obtaining circular indexed nucleic acid fragments, wherein the first strand of said C-indexer is ligated to the fragments to form a closed circular strand, and wherein the second strand of said C-indexer is ligated to the fragments to form a discontinuous strand; (d) amplifying the closed circular strand of said circular indexed nucleic acid fragments by rolling circle amplification. The methods may further comprise step of detecting amplified nucleic acid fragments.

The invention also provides a method of amplifying and detecting circular indexed single-stranded nucleic acid fragments, said method comprises the steps of: (a) incubating a C-indexer with single-stranded nucleic acid fragments to allow selective hybridization between complementary protruding single strand of said C-indexer with the 5′ end and 3′ end of said single-stranded nucleic acid fragments; wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; (b) adding ligase to selectively ligate the first strand of said C-indexer with those single-stranded nucleic acid fragments whose 5′- and 3′-ends are fully complementary, respectively, to the 5′- and 3′-protruding single strands of said C-indexer; (c) obtaining circular indexed nucleic acid fragments, wherein the first strand of said C-indexer is ligated to the fragment to form a closed circular strand, wherein the second strand of the C-indexer is not closed; (d) amplifying the closed circular strand of said circular indexed nucleic acid fragments by rolling circle amplification. The method may further comprise a step of detecting amplified nucleic acid fragments.

DNA polymerases useful in rolling circle replication include DNA polymerases that perform rolling circle replication of primed single-stranded circles. Such polymerases are referred to herein as rolling circle DNA polymerases. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the circular vector. A 5′ to 3′ exonuclease activity, if present, might result in the destruction of the synthesized strand. It is also preferred that DNA polymerases for use in the disclosed method are highly processive. The suitability of a DNA polymerase for use in the disclosed method can be readily determined by assessing its ability to carry out rolling circle replication. Exemplary rolling circle DNA polymerases are bacteriophage .phi.29 DNA polymerase (U.S. Pat. Nos. 5,198,543 and 5,001,050 to Blanco et al.), phage M2 DNA polymerase (Matsumoto et al., Gene 84:247 (1989)), phage .phi.PRD1 DNA polymerase (Jung et al., Proc. Natl. Acad. Sci. USA 84:8287 (1987)), VENT.RTM. DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatteijee et al., Gene 97:13-19 (1991)), PRD1 DNA polymerase (Zhu and Ito, Biochim. Biophys. Acta. 1219:267-276 (1994)), modified T7 DNA polymerase (Tabor and Richardson, J. Biol. Chem. 262:15330-15333 (1987); Tabor and Richardson, J. Biol. Chem. 264:6447-6458 (1989); Sequenase.TM. (U.S. Biochemicals)), and T4 DNA polymerase holoenzyme (Kaboord and Benkovic, Curr. Biol. 5:149-157 (1995)). In some embodiments, phi.29 DNA polymerase is used.

For amplifying circular indexed RNA, the polymerase enzyme is preferably selected from the group consisting of T7 RNA Polymerase, T4 RNA Polymerase, SP6 RNA Polymerase, RNA Polymerase II, RNA Polymerase III, T3 RNA Polymerase and E. coli RNA Polymerase. Closely homologous mutants of the enzymes above, i.e., mutants with greater than about 80% homology, can also be included. It is not necessary to include an RNA Polymerase promoter sequence on the circular oligonucleotide template.

In some embodiments, the amplified nucleic acids are detected by hybridizing RCA products with enzyme-labeled short oligo probes. Amplified nucleic acid can be detected and quantitated by measuring the fluorescent or chemiluminescent signals generated from an enzymatic reaction (such as shown FIG. 8).

1. Modified RCA

The circularized DNA can be amplified with methods known in the art, e.g. PCR and RCA. Primer sequence can be encoded in the fixed sequence region of the C-indexer.

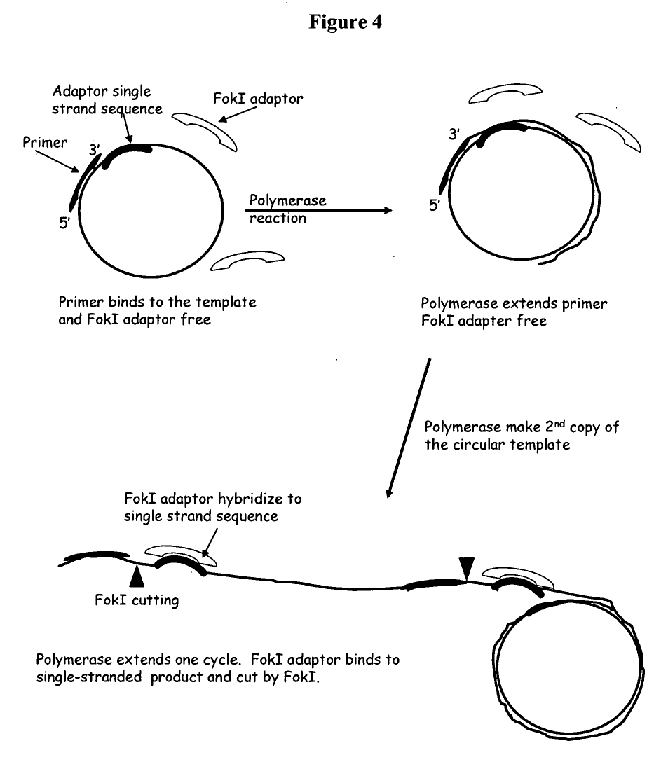

In some embodiments, the invention provides a method, wherein RCA reaction is modified to generate fragmented products of the single-stranded tandem repeats of the circular indexed DNA. The method comprises steps of: (a) mixing a universal endonuclease oligonucleotide adaptor and a RCA primer with circular indexed DNAs, wherein said circular indexed DNAs are formed by using a C-indexer that encodes the sequence of the single-stranded region of said universal endonuclease oligonucleotide adaptor, and wherein the RCA primer hybridizes to the circular DNA template; (b) adding polymerase and type IIS restriction endonuclease to the mixture to start polymerase reaction. After polymerase finishes replicating first copy of the template sequence, it continues to make the second copy and displace the first copy from the template as single stranded DNA (ssDNA). Universal endonuclease oligonucleotide adaptor hybridizes to the ssDNA through its single stranded region and Type IIS restriction endonuclease cleaves the ssDNA. An example of Type IIS restriction endonucleases is FokI. In some embodiments, the universal endonuclease oligonucleotide adaptor is modified at the 3′-end so that it will not be extended by polymerase (FIG. 4).

In a preferred embodiment, ssDNA products from RCA are cut into fragments at the junction of tandem copies of template sequence using universal endonuclease, such as described in Szybalski, 1985 and U.S. Pat. No. 4,935,357. The universal endonuclease utilizes a tailored oligodeoxynucleotide adaptor in conjunction with a Class IIS endonuclease. The fixed sequence region of the C-indexer used in this embodiment encodes for primer sequence and the single-stranded sequence of the oligonucleotide adaptor. The single-stranded sequence of oligonucleotide adaptor is at the 5′ end of the primer-binding site. In this modified RCA reaction, polymerase extends the RCA primer at the presence of a Type IIS endonuclease, preferably FokI, and a universal endonuclease oligonucleotide adaptor containing Fokl recognition at its double stranded region. Complementary strand of the single stranded sequence of the universal endonuclease oligonucleotide adaptor is generated in the polymerase reaction. This sequence, however, is hybridized to the template and is not available to hybridize to the adaptor until polymerase starts to make the second copy and release the first copy as ssDNA. Once it's released, the complementary sequence hybridizes with the oligonucleotide adaptor and FokI cleaves the ssDNA. The single stranded RCA product will be cleaved after each amplification cycle. In this embodiment, 3′ end of the adaptor is modified so that it will not be extended during RCA (FIG. 4).

2. Assemble Double-Stranded Nucleic for Self-Primed Amplification

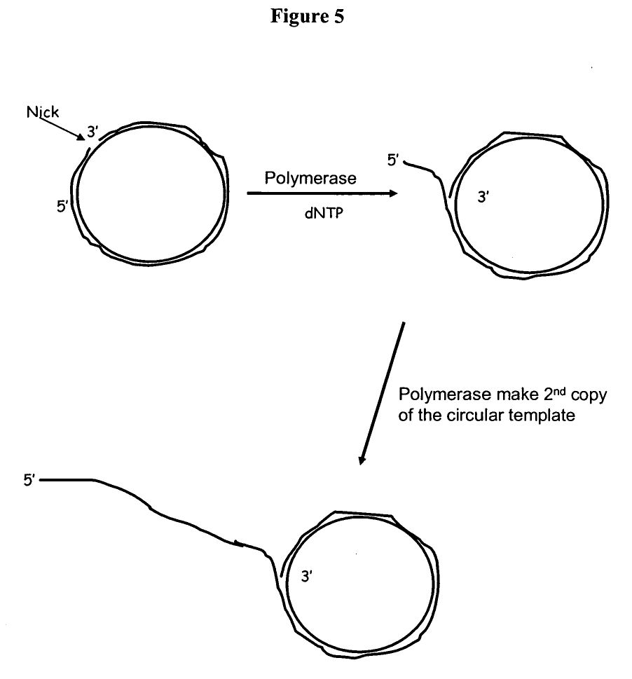

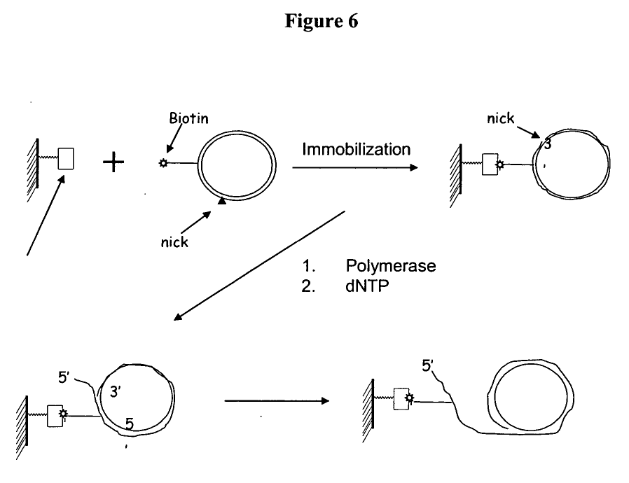

Nucleic acid, preferably DNA, can be can be cleaved into fragments with cohesive ends at specific position using endonuclease restriction enzymes. The C-indexer has two 3′ or 5′ n-base cohesive ends that are completely complementary to the 3′ or 5′ cohesive ends of the DNA fragment containing the DNA target of interest. Only one strand of the said C-indexer is phosphorylated at the 5′ end. When circularizing ds DNA, if the C-indexers are 5′-phosphorylated only in one of the strand, one of the DNA target strands will be circularized while the other strand will have a nick. In such a structure, the nicked strand can serve as primer to amplify the circularized strand. This process is shown in FIG. 5. An affinity tag (biotin, NH₂ etc.) can be encoded into the C-indexers and the amplified nucleic acids can be immobilized. The immobilization process can be performed before amplification and the amplification will be then carried out on the surface (FIG. 6) or after the closed circular strand (the continuous strand or first strand) is separated from the immobilized strand (the discontinuous strand or the second strand).

In some embodiments, the invention provides a method of assemble double-stranded nucleic acid targets, preferably DNA, into a structure in which the target sequence can be amplified without the need of any primer, comprising the steps of: (a) treating nucleic acids, preferably DNA, with one or more restriction endonuclease to generate fragments having at least one 3′ or 5′ n-base protruding single strand (cohesive end), wherein at least one of the fragments contains a DNA target of interest; (b) mixing one or more DNA duplex called C-indexer with said DNA fragment, wherein said C-indexer has two 3′ or 5′ n-base cohesive ends that are completely complementary to the 3′ or 5′ cohesive ends of the DNA fragment containing the target of interest, wherein only one strand of the said C-indexer is phosphorylated at the 5′ end, wherein at sufficiently low DNA concentration and the presence of excessive amount of C-indexers, the cohesive ends of said C-indexers hybridize to the complementary cohesive ends of the DNA fragments to form circular structures; (c) adding DNA ligase to said circular structures, wherein only one strand of the C-indexer has phosphate at the 5′ end, and a nick is generated on the non-phosphorylated stranded of the circular DNAs; (d) adding a polymerase, a mixture of all four deoxynucleotide precursors, and appropriate buffers to the circularized DNA to start the polymerase reaction at the 3′ end of the nick to generate single-stranded nucleic acids containing thousands of tandem copies of the circular template sequence; (e) collecting the amplified sing-stranded DNA products.

The restriction endonucleases can be Type IIS restriction endonuclease (such as FokI, AarI, and HgaI) or interrupted palindrome (IP) restriction endonucleases. N can be any number, such as 3, 4, and 5. The polymerase can be any polymerase known in art for replicate circular nucleic acid template. In some embodiments, said C-indexer is covalently attached to an affinity tag, such as a biotin or a NH₂. In some embodiments, the circularized DNA is immobilized to a surface through said affinity tag before polymerase reaction. In some embodiments, the surface is a plate, a chip or a bead.

3. Assemble Single-Stranded Nucleic for Self-Primed Amplification

A C-indexer can also be used to circularize single-stranded nucleic acid for self-primed amplification. The C-indexer has both 5′ and 3′ cohesive ends (therefore one strand is longer than the other strand as show in FIG. 7). 5′ end of the shorter strand is phosphorylated and sequences of the 5′ and 3′ cohesive ends are complementary to the 5′ and 3′ end of the nucleic acid target respectively. After ligation, the single-stranded nucleic acid is circularized with the shorter stranded of the C-indexer. The longer strand of the C-indexer is hybridized to the single-stranded circular template containing the target and can serve as primer to amplify the single-stranded circular template (FIG. 7).

In some embodiments, the invention provides a method of assemble single-stranded nucleic acid targets into a structure in which the target sequence can be amplified without the need for any primer, comprising the steps of: (a) denaturing double-stranded nucleic acid or cleaving single-stranded nucleic acids to generate fragments, wherein at least one of the fragments contains a nucleic acid target of interest; (b) mixing one or more DNA duplex called C-indexer with said nucleic acid fragment, wherein said C-indexer has both 3′ and 5′ n-base cohesive ends that are completely complementary to the 3′ and 5′ ends of the nucleic acid fragment containing the nucleic acid target of interest, wherein only the recessive strand (shorter strand) of the said C-indexer is phosphorylated at the 5′ end, and wherein at sufficiently low fragment concentration and the presence of excessive amount of C-indexers, the cohesive ends of said C-indexers hybridize to the 5′ and 3′ ends of the nucleic acid fragments to form circular structures; (c) adding ligase to the said circular structures, wherein a single-stranded circular nucleic acid containing the nucleic acid target of interest is generated and hybridized to the protruding strand (the longer strand) of the C-indexer, wherein in the presence of a polymerase, the protruding strand of the C-indexer serve as the primer to duplicate the circular template and generate single-stranded nucleic acids containing thousands of tandem copies of the template sequence; and (d) collecting the amplified sing-stranded nucleic acid products (FIG. 7).

The nucleic acid may be cleaved by any method known in the arts of cleaving single-stranded nucleic acid. For example, nuclease acid may be cleaved by restriction enzyme such as BstN1. N can be any number, such as 3, 4, or 5. The polymerase can be any polymerase known in art for replicate circular nucleic acid template. In some embodiments, said C-indexer is covalently attached to an affinity tag, such as a biotin or NH₂. In some embodiments, the circularized DNA is immobilized to a surface through said affinity tag before polymerase reaction.

4. Isolating and Detecting DNA Targets from a Complex DNA Mixture Without Amplification

In some embodiments, the invention provides a method for isolating and detecting DNA targets from a complex DNA mixture without amplification, comprising the steps of: (a) treating nucleic acids, preferably DNA, with one or more restriction endonuclease to generate fragments having at least one 3′ or 5′ n-base protruding single strand (cohesive end); (b) mixing said C-indexers with said DNA fragment, wherein at sufficiently low DNA concentration and the presence of excessive amount of C-indexers, wherein the cohesive ends of said C-indexers hybridize to the complementary cohesive ends of the DNA fragments to form circular structures, wherein the said C-indexer is phosphorylated only on one of the strand; (c) adding DNA Ligase to the said circular structures to produce double stranded circular DNAs with a nick on one of the strand; (d) immobilizing said double-stranded circular DNAs to a solid surface through an internal affinity moiety covalently attached to the said C-indexers; (e) denaturing the double-stranded circular DNAs and washing away the linear DNAs and the single-stranded circular DNAs in the supernatant; (f) hybridizing a DNA probe that is specifically complementary to the DNA target of interest. The said DNA probe is covalently attached to either a reporter group capable of signal amplification or an affinity moiety that can bind to a reporter group capable of signal amplification; and (g) adding one or more than one substrates to generate signal amplification. The presence of the signal indicates the presence of the DNA target of interest and the amount of the target can be determined by the intensity of the signal. The restriction endonucleases may be Type IIS restriction endonuclease (such as FokI, AarI, and/or HgaI) or interrupted palindrome (IP) restriction endonucleases. N can be any number, such as 3, 4, or 5. The affinity moiety may be a biotin, and the solid surface may be a streptavidin coated magnetic beads. In some embodiments, C-indexer is not phosphorylated on the strand that is covalently attached to the biotin. The nucleic acid can be any DNA, for example, genomic DNA or cDNA (cloned copies of messenger ribonucleic acid), and from any source, such as from human, from biological pathogens or biological agents, or from any cell lines. One example of the method is shown in FIG. 8.

5. Isolating and Genotyping DNA Targets from a Complex DNA Mixture Without Amplification

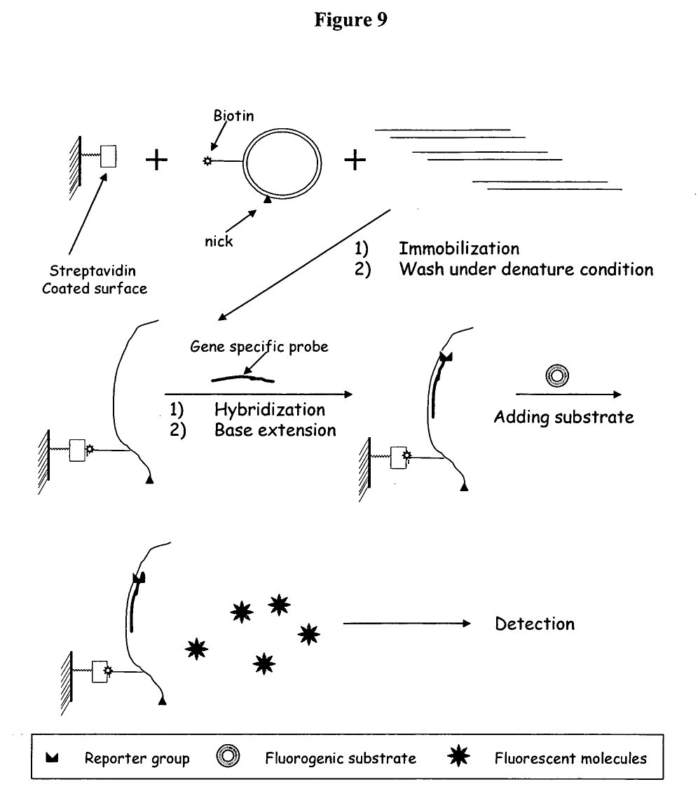

In some embodiments, the invention provides a method for isolating and genotyping DNA targets from a complex DNA mixture without amplification, comprising the steps of: (a) treating nucleic acids, preferably DNA, with one or more restriction endonuclease to generate fragments having at least one 3′ or 5′ n-base protruding single strand (cohesive end); (b) mixing said C-indexers with said DNA fragment, wherein at sufficiently low DNA concentration and the presence of excessive amount of C-indexers, wherein the cohesive ends of said C-indexers hybridize to the complementary cohesive ends of the DNA fragments to form circular structures, and said C-indexer is phosphorylated only on one of the strand; (c) adding DNA Ligase to the said circular structures to produce double stranded circular DNAs with a nick on one of the strand; (d) immobilizing said double-stranded circular DNAs to a solid surface through an internal affinity moiety covalently attached to the said C-indexers; (e) denaturing the double-stranded circular DNAs and washing away the linear DNAs and the single-stranded circular DNAs in the supernatant; (f) hybridizing a DNA probe that is specifically complementary to the DNA target of interest and the 3′ end of the DNA probe hybridizes to the base at the 5′ end of the SNP of interest; and (g) extending the DNA probe by one base through enzymatic reaction using a mixture of two nucleosides complementary to the different SNP variants, wherein two different affinity moieties are covalently linked to the two nucleosides and two affinity moieties bind to two different reporter groups capable of signal amplification; (h) adding one or more than one substrates to generate signal amplification. The presence of the signal indicates the presence of the DNA target of interest and the identity of the signal indicates the genotype of the DNA target. The restriction endonucleases may be Type IIS restriction endonuclease (such as FokI, AarI, and/or HgaI) or interrupted palindrome (IP) restriction endonucleases. N can be any number, such as 3, 4, or 5. The affinity moiety may be a biotin, and the solid surface may be a streptavidin coated magnetic beads. In some embodiments, C-indexer is not phosphorylated on the strand that is covalently attached to the biotin. The nucleic acid can be any DNA, for example, genomic DNA or cDNA (cloned copies of messenger ribonucleic acid), and from any source, such as from human, from biological pathogens or biological agents, or from any cell lines. One example of the method is shown in FIG. 9.

IV. Kits

The invention also provide kits for use in the instant methods. The invention provides a kit for indexing double-stranded nucleic acid fragments from a mixture of double-stranded nucleic acid fragments, said kit comprising: (a) one or more Type IIS or IP restriction endonucleases; (b) a C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of nucleic acid fragments generated by Type IIS or IP restriction endonucleases digestion, wherein said C-indexer is selected from a collection of C-indexers whose 3′- or 5′-protruding single strands collectively encode up to all possible permutations and combinations of nucleotides, A, C, G and T; and (c) ligase and ligase buffer.

The invention also provides a kit for indexing single-stranded nucleic acid fragments from a mixture of single-stranded nucleic acid fragments, said kit comprising: (a) a C-indexer, wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; and (b) ligase and ligase buffer.

In some embodiments, the kits may further comprise a nucleic acid polymerase, a mixture of nucleotide precursors, and a buffer for rolling circle amplification reaction.

The kits may include one or more containers, suitable packaging, and/or instructions for use in accordance with any of the methods of the invention described herein.

1. C-Indexer Circularization Kit

The present invention provides kits and the procedures (instructions) to use the kit for high-level multiplexing single-stranded circular DNA targets preparation. In some embodiments, the kit comprises Type IIS or IP restriction endonucleases; panel of C-indexers; Ligase; ATP; restriction enzyme buffer; ligation buffer; washing buffer, elution buffer; streptavidin coated magnetic beads. In some embodiments, the panel of C-indexers are selected from a set of C-indexers, wherein (a) each member of the set of C-indexers have two 3′- or 5′-cohesive ends of lengths corresponding to the lengths of 3′- or 5′-protruding single strands of the DNA fragments generated by one or more than one Type IIS or IP restriction endonuclease; (b) the 3′- or 5′-cohesive ends of the members of a set of C-indexers collectively encoded up to all permutations and combinations of the nucleotides A, C, G and T; and (c) the members of a set of C-indexers are physically separated from each other on the basis of the identity of their two 3′- or 5′-cohesive ends. In some embodiments, C-indexer is covalently attached to an affinity tag, such as biotin. In some embodiments, the strand of the C-indexer covalently attached to the affinity tag is not phosphorylated.

Procedure of generating single-stranded circular DNA targets using C-indexer circularization kit comprises four parts: restriction digestion; circularization; purification and collection of single-stranded circular DNA targets.

a) Restriction: restriction endonuclease buffer and restriction endonuclease are added into a tube contains DNA sample. The tube is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

c) Purification: after the completion of ligation, streptavidin magnetic beads are added to the tube and mixed with the sample for a specified period of time. Magnet is applied to settle the magnetic beads and supernatant is removed with a pipette. Remove the magnet and add the washing buffer. Mix the beads with the washing buffer before applying the magnet again. Take away the supernatant with a pipette. Repeat the washing step if necessary.

d) Collection of Single-stranded circular DNA targets: elution buffer is added to the tube without magnet. After incubating at a specified temperature for a specified period of time, magnet is applied to settle the beads and the supernatant containing single-stranded circular DNA targets are collected with a pipette.

The C-indexer circularization kit can be used either in a single tube or in a fully automated process for DNA samples placed in microtiter plates.

2. C-Indexer High-Level Multiplexing PCR Kit

The present invention provides the compositions of a kit and a procedure to use the kit to amplify DNA targets with high-level multiplexing PCR. This kit generates double-stranded amplification products. The kit comprises Type IIS or IP restriction endonucleases; panel of C-indexers; Ligase; ATP; restriction enzyme buffer; ligation buffer; washing buffer, elution buffer; streptavidin coated magnetic beads; oligonucleotide primers; DNA polymerase; a mixture of all four deoxynucleotide precursors; PCR buffer and a Thermal Cycler. In some embodiments, the panel of C-indexers are selected from a set of C-indexers, wherein (a) each member of the set of C-indexers have two 3′- or 5′-cohesive ends of lengths corresponding to the lengths of 3′- or 5′-protruding single strands of the DNA fragments generated by one or more than one Type IIS or IP restriction endonuclease; (b) the 3′- or 5′-cohesive ends of the members of a set of C-indexers collectively encoded up to all permutations and combinations of the nucleotides A, C, G and T; (c) the members of a set of C-indexers have the same fixed sequence region; and (d) the members of a set of C-indexers are physically separated from each other on the basis of the identity of their two 3′- or 5′-cohesive ends. In some embodiments, C-indexer is covalently attached to an affinity tag, such as biotin. In some embodiments, the strand of the C-indexer covalently attached to the affinity tag is not phosphorylated.

Procedure for high-level multiplexing PCR of DNA targets using C-indexer high-level multiplexing PCR kit comprising five parts: restriction digestion; circularization; purification; amplification and collection of PCR products.

a) Restriction: restriction endonuclease buffer and restriction endonuclease are added into a tube contains DNA sample. The tube is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

c) Purification: after the completion of ligation, streptavidin magnetic beads are added to the tube and mixed with the sample for a specified period of time. Magnet is applied to settle the magnetic beads and supernatant is removed with a pipette. Remove the magnet and add the washing buffer. Mix the beads with the washing buffer before applying the magnet again. Take away the supernatant with a pipette. Repeat the washing step if necessary.

d) PCR amplification: PCR buffer; oligonucleotide primers; DNA polymerase and a mixture of all four deoxynucleotide precursors are added into the tube with magnetic beads. Place the tube on the Thermal-Cycler to perform PCR reaction using a specified thermal cycling program.

e) Collection of PCR products: place the tube on the magnet to settle the magnetic beads. Supernatant containing the PCR products is collected using a pipette.

The C-indexer high-level multiplexing PCR kit can either be used in a single tube or in a fully automated process to amplify DNA samples placed in microtiter plates.

3. C-Indexer High-Level Multiplexing RCA Kit

The present invention provides a kit and a procedure to use the kit to amplify DNA targets with high-level multiplexing RCA. This kit generates long single-stranded amplification products containing thousands of tandem copies of the complementary sequence of the target DNA. The kit comprises Type IIS or IP restriction endonucleases; panel of C-indexers; Ligase; ATP; restriction enzyme buffer; ligation buffer; washing buffer, elution buffer; streptavidin coated magnetic beads; oligonucleotide primer; DNA polymerase; a mixture of all four deoxynucleotide precursors and RCA buffer. In some embodiments, the panel of C-indexers are selected from a set of C-indexers, wherein (a) each member of the set of C-indexers have two 3′- or 5′-cohesive ends of lengths corresponding to the lengths of 3′- or 5′-protruding single strands of the DNA fragments generated by one or more than one Type IIS or IP restriction endonuclease; (b) the 3′- or 5′-cohesive ends of the members of a set of C-indexers collectively encoded up to all permutations and combinations of the nucleotides A, C, G and T; (c) the members of a set of C-indexers have the same fixed sequence region; and (d) said members of a set of C-indexers are physically separated from each other on the basis of the identity of their two 3′- or 5′-cohesive ends. In some embodiments, C-indexer is covalently attached to an affinity tag, such as biotin. In some embodiments, the strand of the C-indexer covalently attached to the affinity tag is not phosphorylated. In some embodiments, the fixed sequence region of said C-indexers encode sequence of said oligonucleotide primer.

Procedure for high-level multiplexing RCA of DNA targets using C-indexer high-level multiplexing RCA kit comprises five parts: restriction digestion; circularization; purification; amplification and collection of RCA products.

a) Restriction: restriction endonuclease buffer and restriction endonuclease are added into a tube contains DNA sample. The tube is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

f) Purification: after the completion of ligation, streptavidin magnetic beads are added to the tube and mixed with the sample for a specified period of time. Magnet is applied to settle the magnetic beads and supernatant is removed with a pipette. Remove the magnet and add the washing buffer. Mix the beads with the washing buffer before applying the magnet again. Take away the supernatant with a pipette. Repeat the washing step if necessary.

c) RCA amplification: RCA buffer; oligonucleotide primer; DNA polymerase and a mixture of all four deoxynucleotide precursors are added into the tube with magnetic beads. Incubate the tube at a specified temperature for a specified period of time to perform RCA reaction.

d) Collection of RCA products: place the tube on the magnet to settle the magnetic beads. Supernatant containing the RCA products is collected using a pipette.

The C-indexer high-level multiplexing RCA kit can also be used either in a single tube or in a fully automated process to amplify DNA samples placed in microtiter plates.

4. C-Indexer High-Level Multiplexing Modified-RCA Kit

The present invention provides a kit and a procedure to use the kit to amplify DNA targets with high-level multiplexing modified-RCA. This kit generates short single-stranded amplification products. The kit comprises Type IIS or IP restriction endonucleases; panel of C-indexers; Ligase; ATP; restriction enzyme buffer; ligation buffer; washing buffer, elution buffer; streptavidin coated magnetic beads; oligonucleotide primer; DNA polymerase; a mixture of all four deoxynucleotide precursors; RCA buffer; FokI restriction endonuclease and universal restriction endonuclease oligonucleotide adaptor containing FokI recognition site. In some embodiments, the panel of C-indexers are selected from a set of C-indexers, wherein (a) each member of the set of C-indexers have two 3′- or 5′-cohesive ends of lengths corresponding to the lengths of 3′- or 5′-protruding single strands of the DNA fragments generated by one or more than one Type IIS or IP restriction endonuclease; (b) the 3′- or 5′-cohesive ends of the members of a set of C-indexers collectively encoded up to all permutations and combinations of the nucleotides A, C, G and T; (c) the members of a set of C-indexers have the same fixed sequence region encoding primer sequence and the sequence of single-stranded region of the universal restriction endonuclease oligonucleotide adaptor; and (d) said members of a set of C-indexers are physically separated from each other on the basis of the identity of their two 3′- or 5′-cohesive ends. In some embodiments, C-indexer is covalently attached to an affinity tag, such as biotin. In some embodiments, the strand of the C-indexer covalently attached to the affinity tag is not phosphorylated.

Procedure for high-level multiplexing RCA of DNA targets using C-indexer high-level multiplexing modified-RCA kit comprises five parts: restriction digestion; circularization; purification; amplification and collection of RCA products.

a) Restriction: restriction endonuclease buffer and restriction endonuclease are added into a tube contains DNA sample. The tube is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

c) Purification: after the completion of ligation, streptavidin magnetic beads are added to the tube and mixed with the sample for a specified period of time. Magnet is applied to settle the magnetic beads and supernatant is removed with a pipette. Remove the magnet and add the washing buffer. Mix the beads with the washing buffer before applying the magnet again. Take away the supernatant with a pipette. Repeat the washing step if necessary.

d) RCA amplification: RCA buffer; oligonucleotide primer; DNA polymerase; a mixture of all four deoxynucleotide precursors; FokI and oligonucleotide adaptor are added into the tube with magnetic beads. Incubate the tube at a specified temperature for a specified period of time to perform RCA reaction.

e) Collection of RCA products: place the tube on the magnet to settle the magnetic beads. Supernatant containing the RCA products is collected using a pipette.

The C-indexer high-level multiplexing modified-RCA kit can also be used either in a single tube or in a fully automated process to amplify DNA samples placed in microtiter plates.

5. C-Indexer DNA Detection and Genotyping Without DNA Amplification

The present invention provides the procedures and the kits to use C-indexer for DNA detection (FIG. 8) and genotyping (FIG. 9) without any DNA amplification. An assay using the methods or kits of the present invention has multi-levels specificity from ligation reaction, DNA hybridization, and base extension reaction. A specific DNA target can be isolated and enriched from a large amount of sample. The DNA detection and genotyping include the following steps:

a) Restriction: restriction endonuclease buffer and restriction endonuclease are added into a tube contains DNA sample. The tube is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

c) Purification: after the completion of ligation, streptavidin magnetic beads are added to the tube and mixed with the sample for a specified period of time. Double-stranded DNAs are denatured either by adding denaturing reagent or by increasing the temperature. Magnet is applied to settle the magnetic beads and supernatant is removed with a pipette. Remove the magnet and add a washing buffer. Mix the beads with the washing buffer before applying the magnet again. Take away the supernatant with a pipette. Repeat the washing step if necessary. Single-stranded DNA targets are left on the surface.

d) Incorporation of reporter: One or more than one DNA probes that are specifically complementary to the DNA target of interest hybridize with the single-stranded DNA targets attached to the surface. In a DNA detection assay, the DNA probe is covalently attached to either a reporter group capable of signal amplification or an affinity moiety that can bind to a reporter group capable of signal amplification. In a DNA detection and genotyping assay, the 3′ end of the DNA probe hybridizes to the base at the 5′ end of the SNP of interest and is extended one base through enzymatic reaction using a mixture of two nucleosides complementary to the different SNP (single nucleotide polymorphism) variants. Two different affinity moieties are covalently linked to the two nucleosides and two affinity moieties bind to two different reporter groups capable of signal amplification Magnet is applied to settle the magnetic beads and supernatant is removed with a pipette. Remove the magnet and add a washing buffer. Mix the beads with the washing buffer before applying the magnet again. Take away the supernatant with a pipette. Repeat the washing step if necessary.

e) Signal Amplification and Detection: one or more than one signal amplification substrates are added to the tube and incubated at a specific temperature for a specific period of time. The presence of the signal indicates the presence of the DNA target of interest and the intensity of the signal can be used to quantify the amount of the DNA target. In the genotyping assay, different signals are generated for different variants.

In some embodiments, the kit comprise Type IIS or IP restriction endonucleases; one or more C-indexers; ligase; ATP; restriction enzyme buffer; ligation buffer; washing buffer, elution buffer; streptavidin coated magnetic beads; gene specific primer; DNA polymerase; a mixture of all four deoxynucleotide precursors; RCA buffer; a fluorogenic substrate; and a fluorescent molecule. In some embodiments, the gene specific probe comprises a reporter.

6. A Kit for Self-Primed Nucleic Acid Amplification

The present invention provides the compositions of a kit and the procedure to use the kit for high-level multiplexing nucleic acid, preferably DNA, amplification to generate single-stranded DNA targets. The kit is comprised of restriction endonucleases; panel of C-indexers; ligase; ATP; restriction enzyme buffer; ligation buffer; DNA polymerase; a mixture of all four deoxynucleotide precursors, and buffer for polymerase reaction.

Procedure of using this kit comprising three parts: restriction digestion; circularization; and amplification.

a) Restriction: restriction endonuclease buffer and restriction endonuclease are added into a tube contains DNA sample. The tube is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

c) Amplification: DNA polymerase, buffer for polymerase reaction and a mixture of all four deoxynucleotide precursors are added into the tube. Incubate the tube at a specified temperature for a specified period of time to perform polymerase reaction.

The invention also provides a kit and the procedures (instructions) to use the kit for high-level multiplexing nucleic acid amplification to generate single-stranded nucleic acid targets on surface. The kit is comprised of restriction endonucleases; panel of C-indexers covalently attached to an affinity tag; chips or beads that can link with the affinity tag; Ligase; ATP; restriction enzyme buffer; ligation buffer; DNA polymerase; a mixture of all four deoxynucleotide precursors, and buffer for polymerase reaction.

Procedure of using this kit comprising four parts: restriction digestion; circularization; immobilization, and amplification.

a) Restriction: restriction endonuclease buffer and restriction endonuclease are added into DNA sample. The sample is then incubated at a specified temperature for a specific period of time. After restriction digestion, endonuclease is deactivated at elevated temperature and sample is diluted to a specified concentration. The sample is now ready for circularization.

b) Circularization: C-indexers are added to the DNA sample. The mixture is incubated at a specified temperature for a period time to allow complete hybridization between DNA fragments and C-indexers. ATP and Ligase are added to the hybridized mixture. Ligation is carried out at a specified temperature for a specific period of time.

c) Immobilization: Circularized DNA samples are added to surfaces that can link to the affinity tag such as chips and beads.

d) Amplification: DNA polymerase, buffer for polymerase reaction and a mixture of all four deoxynucleotide precursors are added to surface to perform polymerase reaction.

The kits described herein can be used either in a single tube or in a fully automated process to amplify DNA samples placed in microtiter plates. Amplification can also be performed after circularized DNAs are immobilized to surfaces such as chips or beads through an affinity tag (such as biotin or NH₂) covalently attached to the C-indexers.

For simplicity, reference is made generally to DNA with the understanding that these methods and kits apply to any type of nucleic acids described herein.

### EXAMPLES

### Example 1

### DNA Indexing

A commonly used plasmid cloning vector pBR322 is digested with type IIS restriction enzyme Fok I to generated 12 fragments with overhang sequences listed in table 1.

TABLE 1

PBR 322 DNA-FokI Digest
Sorted by 5′ Overhang Sequences

| | 5′ overhang sequence |
|---|---|
| Fragments | 5′ to 3′ |
| Fragment 9 | ACAT/TGTC |
| Fragment 6 | ACCC/CAGA |
| Fragment 2 | ATGC/CCTA |
| Fragment 4 | ATTA/CCTG |
| Fragment 5 | CAGG/GGGT |
| Fragment 8 | CCCG/ATGT |
| Fragment 12 | CTTT/TAAG |
| Fragment 1 | GCAT/CTTA |
| Fragment 10 | GACA/GACA |
| Fragment 3 | TAGG/TAAT |
| Fragment 7 | TCTG/CGGG |
| Fragment 11 | TGTC/AAAG |

Each fragment has unique 5′ and 3′ overhang sequences, therefore different C-indexer is required to index each fragment. A C-indexers is constructed by mixing the following two strands:

| | |
|---|---|
| Strand 1: | |
| 5′ pNNNNGGTCATAGCTGTTTCCTG 3′ | (SEQ ID NO: 1) |
| Strand 2: | |
| 5′ biotinMMMMCAGGAAACAGCTATGACC3′ | (SEQ ID NO: 2) |

Where NNNN and MMMM are the complementary sequences to the 3′ and 5′ overhangs of the fragment to be indexed, respectively. Sequence 5′ GGTCATAGCTGTTTCCTG 3′ (SEQ ID NO: 3) and 5′ CAGGAAACAGCTATGACC 3′ (SEQ ID NO: 4) are the M13 reverse primer and its complementary sequence, respectively.

PBR322 FokI digest is diluted to very low concentration with T4 DNA Ligase buffer. C-indexer (s) and T4 DNA ligase are then added to circularize PBR322 fragment(s). After the ligation, Phil29 polymerase and dNTP mixture are added either directly to the ligation product or after circularized fragment(s) being immobilized to strepavidin coated magnetic beads or plate to amplify the circularized fragment(s) through RCA.

The RCA product(s) in the solution is detected by hybridizing to a detection probe(s) specific to that fragment(s) and visualized in a gel shift assay. Gel shift is only observed for the probe(s) specific to the fragment(s) being indexed. The RCA product(s) on the magnetic beads or plate is detected by measuring the fluorescence signal on the beads or plate after hybridizing the RCA product(s) to the fluorescent-labeled detection probe(s). When more than one fragment are indexed simultaneously, each detection probe is labeled with a different fluorescent dyes. By measuring the fluorescence polarization decrease of the fluorescent-labeled detection probe(s), the amplification of the fragment(s) can be detected in real-time.

The following Table 2 are examples of the C-indexer and detection probe sequences. All the sequences are chemically synthesized.

TABLE 2
Sequences of C-indexer and detection probe

| | |
|---|---|
| C-indexer for fragment 8: | |
| Strand 1: | |
| 5′ pGGGCGGTCATAGCTGTTTGCTG 3′ | (SEQ ID NO: 5) |
| Strand 2: | |
| 5′ biotinACATCAGGAAACAGCTATGACC3′ | (SEQ ID NO: 6) |
| Wherein X = PO₄²⁻ Y = biotin | |
| Detection probe for fragment 8: | |
| 5′ GAC ATT AAC GCT TCT GG 3′ | (SEQ ID NO: 7) |
| C-indexer for fragment 11: | |
| Strand 1: | |
| 5′ pGACAGGTCATAGCTGTTTCCTG 3′ | (SEQ ID NO: 8) |
| Strand 2: | |
| 5′ biotinCTTTCAGGAAACAGCTATGACC3′ | (SEQ ID NO: 9) |
| Wherein X = PO₄²⁻ Y = biotin | |
| Detection probe for fragment 11: | |
| 5′ GTG CTG CAA TGA TAC CG 3′ | (SEQ ID NO: 10) |
| C-indexer for fragment 12: | |
| Strand 1: | |
| 5′ pAAAGGGTCATAGCTGTTTCCTG 3′ | (SEQ ID NO: 11) |
| Strand 2: | |
| 5′ biotinCTTACAGGAAACAGCTATGACC3′ | (SEQ ID NO: 12) |
| | (SEQ ID NO: 12) |
| Wherein X = PO₄²⁻ Y = biotin | |
| Detection probe for fragment 12: | |
| 5′ GAG TAA GTA GTT CGC CAG 3′ | (SEQ ID NO: 13) |

### Example 2

### Sequence Specific Circularize and Amplification of Single-Stranded DNA

Oligonucleotides with the following sequences are chemically synthesized to construct the C-indexer to circularize single-stranded pBR322 fragment.

| | |
|---|---|
| Strand 1: | |
| 5′ pCACAATTCCACACAAC 3′ | (SEQ ID NO: 14) |
| Strand 2: | |
| 5′ biotin NNNNGTTGTGTGG | (SEQ ID NO: 15) |
| AATTGTGMMMM 3′ | |

Where NNNN and MMMM are the complementary sequences to the 3′ and 5′ end sequence of the fragment to be circularized, respectively.

PBR322 FokI digest is diluted to very low concentration with T4 DNA Ligase buffer. The double-stranded DNAs are denatured by incubating at 94° C. for 5 min and quickly cool on ice. C-indexer (s) and T4 DNA ligase are then added to circularize PBR322 fragment(s). After the ligation, Phil29 polymerase and dNTP mixture are added either directly to the ligation product or after circularized fragment(s) being immobilized to strepavidin coated magnetic beads or plate to amplify the circularized fragment(s) through RCA.

The RCA product(s) in the solution is detected by hybridizing to a detection probe(s) specific to that fragment(s) and visualized in a gel shift assay. Gel shift is only observed for the probe(s) specific to the fragment(s) being indexed. The RCA product(s) on the magnetic beads or plate is detected by measuring the fluorescence signal on the beads or plate after hybridizing the RCA product(s) to the fluorescent-labeled detection probe(s). When more than one fragment are indexed simultaneously, each detection probe is labeled with a different fluorescent dyes. By measuring the fluorescence polarization decrease of the fluorescent-labeled detection probe(s), the amplification of the fragment(s) can be detected in real-time.

The following are examples of the C-indexer and detection probe sequences. All the sequences are chemically synthesized. These C-indexers are designed to circularize the sense strand of the FokI digest.

TABLE 3

Examples of the C-indexer
and detection probe sequences

| | |
|---|---|
| C-indexer for fragment 8: | |
| Strand 1: | |
| 5′ XCACAATTCCACACAAC 3′ | (SEQ ID NO: 16) |
| Strand 2: | |
| 3′ CGATACGGGGTGTTAAGGTGTGTTGCTGC | (SEQ ID NO: 17) |
| CTGTTCY 5′ | |
| Wherein X = PO₄²⁻ Y = biotin | |
| Detection probe for fragment 8: | |
| 5′ GACATTAACGCTTCTGG 3′ | (SEQ ID NO: 18) |
| C-indexer for fragment 11: | |
| Strand 1: | |
| 5′ XCACAATTCCACACAAC 3′ | (SEQ ID NO: 19) |
| Strand 2: | |
| 3′ CGAAATAGACAGTGTTAAGGTGTGTTGTT | (SEQ ID NO: 20) |
| GCAGGACCY5′ | |
| Wherein X = PO₄²⁻ Y = biotin | |
| Detection probe for fragment 11: | |
| 5′ GTGCTGCAATGATACCG 3′ | (SEQ ID NO: 21) |
| C-indexer for fragment 12: | |
| Strand 1: | |
| 5′ XCACAATTCCACACAAC 3′ | (SEQ ID NO: 22) |
| Strand 2: | |
| 3′ GCGGATAAAGGTGTTAAGGTGTGTTGAGA | (SEQ ID NO: 23) |
| ATTATGCAGY5′ | |
| Wherein X = PO₄²⁻ Y = biotin | |
| Detection probe for fragment 12: | |
| 5′ GAGTAAGTAGTTCGCCAG 3′ | (SEQ ID NO: 24) |

### Example 3

### Circularize and Amplification of Single-Stranded Oligonucleotide

Three 5′ phosphorylated SARS-specific probes and their corresponding C-indexers (Table 4) are chemically synthesized.

TABLE 4

Sequences of SARS-specific probes,
C-indexers, and detection probes

| | |
|---|---|
| Cor-p-F2 (+) | |
| 5′X CTAACATGCTTAGGATAATGG 3′ | (SEQ ID NO: 25) |
| Strand 1: | |
| 5′ XCACAATTCCACACAAC 3′ | (SEQ ID NO: 26) |
| Strand 2: | |
| 3′ TTACCGTGTTAAGGTGTGTTGGATTGY 5′ | (SEQ ID NO: 27) |
| Wherein X = PO₄²⁻ Y = biotin | |
| Detection probe for Cor-p-F2 (+): | |
| 5′ X GTTTCCTGCTAACATG 3′ | (SEQ ID NO: 28) |
| Cor-p-F3 (+) | |
| 5′ GCCTCTCTTGTTCTTGCTCGC 3′ | (SEQ ID NO: 29) |
| Strand 1: | |
| 5′ XCACAATTCCACACAAC 3′ | (SEQ ID NO: 30) |
| Strand 2: | |
| 3′ GAGCGGTGTTAAGGTGTGTTGCGGAGY 5′ | (SEQ ID NO: 31) |
| Wherein X = PO₄²⁻ Y = biotin | |
| Detection probe for Cor-p-F3 (+): | |
| 5′ GCTCGCGGTCATAGC 3′ | (SEQ ID NO: 32) |
| Cor-p-R1 (−) | |
| 5′X CAG GTA AGC GTA AAA CTC ATC 3′ | (SEQ ID NO: 33) |
| Strand 1: | |
| 5′ XCACAATTCCACACAAC 3′ | (SEQ ID NO: 34) |
| Strand 2: | |
| 3′ AGTAGGTGTTAAGGTGTGTTGGTCCAY 5′ | (SEQ ID NO: 35) |
| Wherein X = PO₄²⁻ Y = biotin | |
| Detection probe for Cor-p-R1 (−): | |
| 5′ CTCATCGGTCATAGC 3′ | (SEQ ID NO: 36) |

Mixing the probe at very diluted concentration with the corresponding C-indexer in the presence of T4 DNA ligase circularizes the probe with the C-indexer. After the ligation, Phil29 polymerase and dNTP mixture are added either directly to the ligation product or after circularized DNA being immobilized to strepavidin coated magnetic beads or plate to amplify the circularized probe through RCA.

The RCA product in the solution is detected by hybridizing to the detection probe, which is designed to hybridize at the junction of between SARS specific probe and the C-indexer, and visualized by a gel shift assay. Gel shift is only observed when the probe is hybridized to the long single-stranded RCA product containing tandem copies of the SARS specific probe. The RCA product on the magnetic beads or plate is detected by measuring the fluorescence signal on the beads or plate after hybridizing the RCA product to the fluorescent-labeled detection probe.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

### List of References

### U.S. Patent Documents

| | | |
|---|---|---|
| 6,013,445 | January 2000 | Albrecht, et al. |
| 5,552,278 | September 1996 | Brenner |
| 5,599,675 | February 1997 | Brenner |
| 5,856,093 | January 1999 | Brenner |
| 5,831,065 | November 1998 | Brenner |
| 5,714,330 | February 1998 | Brenner, et al. |
| 5,503,980 | April 1996 | Cantor |
| 5,508,169 | April 1996 | Deugau et al. |
| 6,175,002 | January 2001 | DuBridge, et al. |
| 5,888,737 | March 1999 | DuBridge, et al. |
| 6,280,948 | August 2001 | Guilfoyle, et al. |
| 6,228,999 | May 2001 | Guilfoyle, et al. |
| 5,994,068 | November 1999 | Guilfoyle, et al. |
| 5,707,807 | January 1998 | Kato |
| 4,683,202 | July 1987 | Mullis |
| 6,284,497 | September 2001 | Sabanayagam, et al. |
| 4,935,357 | June 1990 | Szybalski |
| 4,321,365 | March 1982 | Wu et al. |
| 6,344,329 | February 2002 | Lizardi |

### Other References

Brenner, S. & Livak, K. J. (1989) Proc. Natl. Acad. Sci. USA, 86:8902-6.

Cohen, S. N. et al. (1973) Proc. Natl. Acad. Sci. USA, 70: 3240.

Fire, A., Xu, S-Q (1995) Proc. Natl. Acad. Sci. USA, 92: 4641-45.

Guilfoyle, R. A. et al. (1997) Nucleic Acids Research, 25(9): 1854-58.

Kessler, C. et al. (1985) Gene, 33: 1-102.

Lander, E. S. (2001) Nature, 409: 860-921.

Lizardi, PM et al. (1998) Nat. Genet., 19: 225-232.

Liu, D. et al. (1996) J. Am. Chem. Soc., 118: 1587-94.

Murray, N. E. and Murray, K. (1974) Nature 251: 476.

Mullis, K. B. & Faloona, F. A. (1987) Methods Enzymol. 155: 335-350.

Nallur, G. et al. (2001) Nucleic Acids Research, 29(23): e118.

Nilsson, M. et al. (1997) Nat. Genet., 16: 252-255.

Saiki, R. K. et al. (1985) Science 230: 1350-1354.

Schweitzer, B. et al. (2000) Proc. Natl. Acad. Sci. USA, 97: 10113-10119.

Sibson R. D. & Gibbs, F. E. M. (2001) Nucleic Acids Research, 29(19): e95.

Smith, D. R. (1992) PCR Methods and Applications, 2: 21-27.

Szybalski, W. et al. (1991) Gene, 100: 13-26.

Szybalski, W. (1985) Gene, 40: 169-173.

Tanaka, T. and Weisblum, B. (1975) J. Bacteriology 121: 354.

Thomas, M. et al., (1974) Proc. Natl. Acad. Sci. USA, 71: 4579.

Unrau, Paul, et al., (1994) Gene, 145:163-169.

Venter, J. C. et al., (2001) Science, 291: 1304-51.

Zhang, D. Y. et al., (1998) Gene, 211: 277-285.

Zheleznaya, L. A., et al. (1995) Biochemistry (Moscow), 60: 1037-1043.

Zhong, X-B et al., (2001) Proc. Natl. Acad. Sci. USA, 98: 3940-45.

The claimed invention is:

1. A method for indexing double-stranded nucleic acid fragments from a mixture of double-stranded nucleic acid fragments, said method comprises the steps of:
(a) treating nucleic acids with one or more restriction endonucleases selected from the group consisting of Type IIS restriction endonuclease and interrupted palindrome (IP) restriction endonuclease to generate fragments having at least one 3′- or 5′-protruding single strand;
(b) incubating a C-indexer with said fragments generated in step (a) to allow selective hybridization between complementary protruding single strand of said fragments and said C-indexers, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of the fragments, wherein said C-indexer is selected from a collection of C-indexers whose 3′- or 5′-protruding single strands collectively encode up to all possible permutations and combinations of nucleotides, A, C, G and T;
(c) adding ligase to selectively ligate said C-indexer to those fragments whose 3′- or 5′-protruding single strands are fully complementary to the 3′- or 5′-protruding protruding single strands of said C-indexer; and
(d) obtaining circular indexed nucleic acid fragments, wherein the first strand of said C-indexer is ligated to the fragments to form a closed circular strand, and wherein the second strand of said C-indexer is ligated to the fragments to form a discontinuous strand.

2. The method of claim 1, wherein the length of the 3′- or 5′-protruding single strand of said C-indexer is longer than 2 bases.

3. The method of claim 2, the length of the 3′- or 5′-protruding single strand of said C-indexer is 3, 4, or 5 bases.

4. The method of claim 1, wherein the restriction endonuclease is a Type IIS restriction endonuclease.

5. The method of claim 4, wherein the Type IIS restriction endonuclease is selected from the group consisting of FokI, AarI, and HgaI.

6. The method of claim 1, wherein the restriction endonuclease is interrupted palindrome (IP) restriction endonuclease.

7. The method of claim 1, wherein the 5′ end of the second strand of said C-indexer cannot be ligated.

8. The method of claim 1, wherein the second strand includes an affinity tag covalently attached to said C-indexer.

9. The method of claim 8, wherein the affinity tag is selected from a group consisting of a biotin moiety, a digoxigenin (DIG), a reactive amine, thiol group, an antibody, an antigen or oligonucleotide.

10. The method of claim 8, further comprising steps of:
(e) immobilizing said circular indexed nucleic acid fragments to a surface treated with an affinity target capable of binding said affinity tag through the affinity tag;
(f) washing away linear nucleic acid fragments;
(g) denaturing the closed circular strand and the discontinuous strand of the circular indexed fragments to release the closed circular strand from the solid surface;
(h) collecting the released closed circular strand of the circular indexed fragments.

11. The method of claim 10, wherein the surface is a plastic surface, a glass surface or a membrane surface.

12. A method of amplifying circular indexed nucleic acid fragments, said method comprises the steps of:
(a) treating nucleic acids with one or more restriction endonucleases selected from the group consisting of Type IIS restriction endonuclease and interrupted palindrome (IP) restriction endonuclease to generate fragments having at least one 3′- or 5′-protruding single strand;
(b) incubating a C-indexer with said fragments generated in step (a) to allow selective hybridization between complementary protruding single strand of said fragments and said C-indexers, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of the fragments, wherein said C-indexer is selected from a collection of C-indexers whose 3′- or 5′-protruding single strands collectively encode up to all possible permutations and combinations of nucleotides, A, C, G and T;
(c) adding ligase to selectively ligate said C-indexer to those fragments whose 3′- or 5′-protruding single strands are fully complementary to the 3′- or 5′-protruding protruding single strands of said C-indexer;
(d) obtaining circular indexed nucleic acid fragments, wherein the first strand of said C-indexer is ligated to the fragments to form a closed circular strand, and wherein the second strand of said C-indexer is ligated to the fragments to form a discontinuous strand; and
(e) amplifying the closed strand of said circular indexed nucleic acid fragments by rolling circle amplification.

13. The method of claim 12, wherein the length of the 3′- or 5′-protruding single strand of said C-indexer is longer than 2 bases.

14. The method of claim 13, the length of the 3′- or 5′-protruding single strand of said C-indexer is 3, 4, or 5 bases.

15. The method of claim 12, wherein the restriction endonuclease is a Type IIS restriction endonuclease.

16. The method of claim 15, wherein the Type IIS restriction endonuclease is selected from the group consisting of FokI, AarI, and HgaI.

17. The method of claim 12, wherein the restriction endonuclease is interrupted palindrome (IP) restriction endonuclease.

18. The method of claim 12, wherein the 5′ end of the second strand of said C-indexer cannot be ligated.

19. The method of claim 12, wherein said discontinuous strand is used as rolling circle amplification primer.

20. The method of claim 12, wherein the second strand of said C-indexer is covalently attached to an affinity tag.

21. The method of claim 20, wherein the affinity tag is selected from a group consisting of a biotin moiety, a digoxigenin (DIG), a reactive amine, thiol group, an antibody, an antigen or oligonucleotide.

22. The method of claim 20, wherein the circular indexed nucleic acid fragments are immobilized to a surface treated with an affinity target capable of binding said affinity tag through said affinity tag before said rolling circle amplification.

23. The method of claim 22, wherein the surface is plastic surface, glass surface or membrane surface

24. The method of claim 22, wherein said rolling circle amplification is performed on the surface.

25. The method of claim 22, wherein said rolling circle amplification is performed after said closed circular strand is separated from said discontinuous strand.

26. A kit for indexing double-stranded nucleic acid fragments from a mixture of double-stranded nucleic acid fragments, said kit comprising: (a) one or more Type IIS or IP restriction/endonucleases; (b) a C-indexer, wherein said C-indexer is a double-stranded linear nucleic acid comprising a first strand and a second strand and a 3′- or a 5′-protruding single strand on said first strand and said second strand, wherein the lengths of the 3′- or 5′-protruding single strands of said C-indexer correspond to the lengths of the 3′- or 5′-protruding single strands of nucleic acid fragments generated by Type IIS or IP restriction endonucleases digestion, wherein said C-indexer is selected from a collection of C-indexers whose 3′- or 5′-protruding single strands collectively encode up to all possible permutations and combinations of nucleotides, A, C, G and T; and (c) ligase and ligase buffer.

27. The kit of claim 26, wherein the 5′ end of the second strand of said C-indexer is not phosphorylated.

28. The kit of claim 26, wherein the second strand of said C-indexer is covalently attached to an affinity tag.

29. The kit of claim 28, further comprising a surface treated with an affinity target capable of binding said affinity tag.

30. The kit of claim 26, further comprising a DNA polymerase, a mixture of all four deoxynucleotide precursors, and a rolling circle amplification buffer.

31. A method for indexing single-stranded nucleic acid fragments from a mixture of single-stranded nucleic acid fragments, said method comprises the steps of:
(a) incubating a C-indexer with single-stranded nucleic acid fragments to allow selective hybridization between complementary protruding single strands of said C-indexer with the 5′ end and 3′ end of said single-stranded nucleic acid fragments; wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T;
(b) adding ligase to selectively ligate said C-indexer with those single-stranded nucleic acid fragments whose 5′- and 3′-ends are fully complementary, respectively, to the 5′- and 3′-protruding single strands of said C-indexer; and
(c) obtaining circular indexed nucleic acid fragments, wherein the first strand of said C-indexer is ligated to the fragments to form a closed circular strand, wherein the second strand of the C-indexer is not closed.

32. The method of claim 31, wherein the lengths of the 5′- and 3′-protruding single strand on the second strand of said C-indexer are the same.

33. The method of claim 31, wherein the lengths of the 5′- and 3′-protruding single strand on the second strand of said C-indexer are different.

34. The method of claim 31, wherein the lengths of the 5′- and 3′-protruding single strand on the second strand of said C-indexer are between 3 and 30.

35. The method of claim 31, wherein the 5′ end of the second strand of said C-indexer cannot be ligated.

36. The method of claim 31, wherein the second strand includes an affinity tag covalently attached to said C-indexer.

37. The method of claim 36, wherein the affinity tag is selected from a group consisting of a biotin moiety, a digoxigenin (DIG), a reactive amine, a thiol group, an antibody, an antigen or oligonucleotide.

38. The method of claim 31, wherein the single-stranded nucleic acids can be generated from denaturing double-stranded nucleic acids.

39. A method of amplifying circular indexed single-stranded nucleic acid fragments, said method comprises the steps of:
(a) incubating a C-indexer with single-stranded nucleic acid fragments to allow selective hybridization between complementary protruding single strands of said C-indexer with the 5′ end and 3′ end of said single-stranded nucleic acid fragments; wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T;
(b) adding ligase to selectively ligate the first strand of said C-indexer with those single-stranded nucleic acid fragments whose 5′- and 3′-ends are fully complementary, respectively, to the 5′- and 3′-protruding single strands of said C-indexer;
(c) obtaining circular indexed nucleic acid fragments, wherein the first strand of said C-indexer is ligated to the nucleic acid fragments to form a closed circular strand, wherein the second strand of the C-indexer is not closed; and
(d) amplifying the closed strand of said circular indexed nucleic acid fragments by rolling circle amplification.

40. The method of claim 39, wherein the lengths of the 5′- and 3′-protruding single strand on the second strand of said C-indexer are the same.

41. The method of claim 39, wherein the lengths of the 5′- and 3′-protruding single strand on the second strand of said C-indexer are different.

42. The method of claim 39, wherein the lengths of the 5′- and 3′-protruding single strand on the second strand of said C-indexer are between 3 and 30.

43. The method of claim 39, wherein the 5′ end of the second strand of said C-indexer cannot be ligated.

44. The method of claim 39, wherein the single-stranded nucleic acids can be generated from denaturing double-stranded nucleic acids.

45. The method of claim 39, wherein said second strand of the C-indexer is used as rolling circle amplification primer.

46. The method of claim 39, wherein the second strand of said C-indexer is covalently attached to an affinity tag.

47. The method of claim 46, wherein the affinity tag is selected from a group consisting of a biotin moiety, a digoxigenin (DIG), a reactive amine, a thiol group, an antibody, an antigen or oligonucleotide.

48. The method of claim 46, wherein the circular indexed nucleic acid fragments are immobilized to a surface treated with an affinity target capable of binding to said affinity tag through said affinity tag before said rolling circle amplification.

49. The method of claim 48, wherein said surface is a plastic surface, glass surface or membrane surface

50. The method of claim 48, wherein said rolling circle amplification is performed on the surface.

51. The method of claim 48, wherein said rolling circle amplification is performed after said closed circular strand is separated from said second strand of the C-indexer.

52. A kit for indexing single-stranded nucleic acid fragments from a mixture of single-stranded nucleic acid fragments, said kit comprising: (a) a C-indexer, wherein said C-indexer is a double-stranded linear nucleic acid comprising a first strand and a second strand and a 5′- and a 3′-protruding single strand on said second strand, wherein said C-indexer is selected from a collection of C-indexers whose 5′- and 3′-protruding single strands collectively encode up to all possible permutations and combinations of nucleotides, A, C, G and T; and (b) ligase and ligase buffer.

53. The kit of claim 52, wherein the 5′ end of the second strand of said C-indexer is not phosphorylated.

54. The kit of claim 52, further comprising a nucleic acid polymerase, a mixture of nucleotide precursors, and a buffer for rolling circle amplification reaction.

55. The kit of claim 52, wherein one of the strands of said C-indexer is covalently attached to an affinity tag.

56. The kit of claim 55, wherein the affinity tag is selected from a group consisting of a biotin moiety, a digoxigenin (DIG), a reactive amine, a thiol group, an antibody, an antigen or oligonucleotide.

57. The kit of claim 56, further comprising a surface treated with an affinity target capable of binding said affinity tag.

58. The kit of claim 52, wherein the single-stranded nucleic acids can be generated by denaturing double-stranded nucleic acids.
